(19) United States
(12) Patent Application Publication
(10) Pub. No.: US 2026/0364104 A1
(43) Pub. Date: Nov. 19, 2026

(54) BIOREDUCIBLE POLYMER AND USE THEREOF

(71) Applicant: The Johns Hopkins University, Baltimore, MD (US)

(72) Inventors: Jung Soo Suk, Clarksville, MD (US); Gijung Kwak, Baltimore, MD (US); Kai Zhang, Hangzhou (CN)

(21) Appl. No.: 19/108,139

(22) PCT Filed: Aug. 30, 2023
(86) PCT No.: PCT/US2023/031502

§ 371 Date: Feb. 28, 2025

Related U.S. Application Data
(60) Provisional application No. 63/373,971, filed on Aug. 30, 2022.

(51) Int. Cl. A61K 31/795; A61K 9/51; A61K 31/7105; A61K 38/19

(52) U.S. Cl. CPC A61K 31/795; A61K 9/5146; A61K 31/7105; A61K 38/195

(57) ABSTRACT

Provided herein, inter alia, a composition or an engineered gene delivery composition including nanoparticles and polymers (e.g., poly(disulfide amine) or PDSA polymers) containing chemically distinct functional side chain groups and different amounts of bioreducible moieties. Also provided is an engineered PEGylated nanoparticles including polymers (e.g., PDSA polymers).

### CROSS-REFERENCES TO RELATED APPLICATIONS

This application claims the benefit of priority of U.S. Provisional Application No. 63/373,971 filed on Aug. 30, 2022, which is incorporated herein by reference in its entirety and for all purposes.

### STATEMENT OF GOVERNMENT SUPPORT

This invention was made with government support under grant number DK132425 awarded by the National Institutes of Health. The government has certain rights in the invention.

### TECHNICAL FIELD

Provided herein, inter alia, are a bioreducible polymer and its use for delivery of nucleic acid-based medicines to a target, e.g., to eyes and beyond.

### BACKGROUND

Gene therapy has emerged as a state-of-the-art breakthrough therapy for treating patients with vision impairment or irreversible vision loss. The current mainstay gene delivery approach involves injection of adeno-associated virus (AAV) into the subretinal space, including the first and only FDA-approved ocular gene therapy product developed for treating a rare inherited retinal disease (i.e., Luxterma®) and multiple pipelines under clinical investigation. AAV has been the gene vector of choice due to its inherent ability to infect cells with relatively minimal pathologic risk. However, AAV holds several shortcomings, including limited packaging capacity, clinically reported intraocular inflammation and therapy-inactivating immunogenicity. The associated cost is enormous, which would be a greater concern for its broader applications to more commonly occurring acquired retinal disorders. In addition, the subretinal injection procedure, employed to gain direct access of gene vectors to target retinal cells, comes with adverse effects, including those necessitating an additional surgery, and results in limited topographic therapeutic coverage near the injection site. Thus, there is a demand for developing a gene delivery strategy that addresses these limitations of the current approach.

### SUMMARY

Provided herein, inter alia, a composition or an engineered gene delivery composition including nanoparticles and polymers (e.g., poly(disulfide amine) or PDSA polymers) containing chemically distinct functional side chain groups and different amounts of bioreducible moieties. Also provided is an engineered PEGylated nanoparticles including polymers (e.g., PDSA polymers).

In one aspect, the compositions comprise one or more polymers that comprise a poly(disulfide amine) structure.

In an aspect, the compositions comprise one or more polymers that comprise a structure of any Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein, or its subordinates, or an isomer, metabolite, prodrug, hydrate, or pharmaceutically acceptable salt thereof, or a device including the polymers or the compositions.

In an aspect, the disclosure provides polymers (e.g., PDSA polymers or a polymer comprise a structure of any one of Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein) including or made of monomers, which are chemically linked via reducible bonds (e.g., disulfide bonds) or include reducible bond (e.g., disulfide bonds). The polymer preferably may facilitate release in the reducing intracellular environment and promote clearance of carrier materials. In addition, the PDSA polymer may provide non-viral gene delivery in transfecting cell, particularly, retinal cells as well as safety profile.

In an aspect, the disclosure provides use of the gene delivery composition. For example, the composition may be optimized for retinal cell transfection and safety in vitro, or for ability to penetrate bovine vitreous and ILM ex vivo. Preferably, the PDSA polymers in the composition may facilitate intracellular trafficking of plasmid payloads by promoting endosomal escape and plasmid release after endocytic uptake by retinal cells. In addition, the gene delivery composition may efficiently penetrate key extracellular barriers, including vitreous gel and ILM.

In an aspect, the disclosure provides the composition for less invasive intravitreal administration. For example, the composition may efficiently traverse the key extracellular barriers, e.g., vitreous gel and inner limiting membrane (ILM), by applying non-adhesive surface coatings including polyethylene glycol (PEG).

Provided is, in various embodiments, a pharmaceutical composition comprising a poly(disulfide amine) polymer and/or a polymer of any one of Formulae.

In certain aspect, the polymer comprises or has a structure of Formula (I),

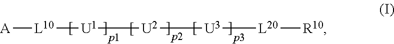

or an isomer, metabolite, prodrug, hydrate, or pharmaceutically acceptable salt thereof,
wherein:
A is a substituted or unsubstituted alkyl, substituted or unsubstituted cycloalkyl, substituted or unsubstituted heteroalkyl, substituted or unsubstituted aryl, or a substituted or unsubstituted heteroaryl,
L¹⁰ is -L^{10A}-L^{10B}-L^{10C}-L^{10D}-L^{10E}-, and at least one of L^{10A}, L^{10B}, L^{10C}, L^{10D}, and L^{10E} is not a bond;
L²⁰ is -L^{20A}-L^{20B}-L^{20C}-L^{20D}-L^{20E}-, and at least one of L^{20A}, L^{20B}, L^{20C}, L^{20D}, and L^{20E} is not a bond;
R¹⁰ is hydrogen, halogen, —CX¹⁰₃, —CHX¹⁰₂, —CH₂X¹⁰, —SOₙ₁₀R^{10D}, —SOᵥ₁₀NR^{10A}R^{10B}, —NHNR^{10A}R^{10B}, —ONR^{10A}R^{10B}, —NHC═(O)NHNR^{10A}R^{10B}, —NHC(O)NR^{10A}R^{10B}, —NR^{10A}R^{10B}, C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —OCX¹⁰₃, —OCHX¹⁰₂, —OCH₂X¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
each p1, p2, and p3 is independently an integer from 0 to 100, and at least one of p1, p2, and p3 is not 0;
U¹ has a structure of Formula (A),

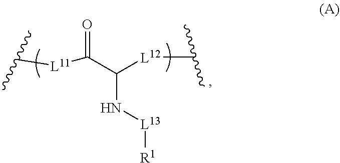

wherein:
L¹¹ is -L^{11A}-L^{11B}-L^{11C}-L^{11D}-L^{11E}-, and at least one of L^{11A}, L^{11B}, L^{11C}, L^{11D}, and L^{11E} is not a bond;
L¹² is -L^{12A}-L^{12B}-L^{12C}-L^{12D}-L^{12E}-, and at least one of L^{12A}, L^{12B}, L^{12C}, L^{12D}, and L^{12E} is not a bond;
L¹³ is -L^{13A}-L^{13B}-L^{13C}-L^{13D}-L^{13E}-, and at least one of L^{13A}, L^{13B}, L^{13C}, L^{13D}, and L^{13E} is not a bond; and
R¹ is hydrogen, halogen, —CX¹₃, —CHX¹₂, —CH₂X¹, —SOₙ₁R^{1D}, —SOᵥ₁NR^{1A}R^{1B}, —NHNR^{1A}R^{1B}, —ONR^{1A}R^{1B}, —NHC—(O)NHNR^{1A}R^{1B}, —NHC(O)NR^{1A}R^{1B}, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
U² has a structure of Formula (B),

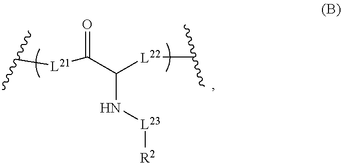

wherein:
L²¹ is -L^{21A}-L^{21B}-L^{21C}-L^{21D}-L^{21E}-, and at least one of L^{21A}, L^{21B}, L^{21C}, L^{21D}, and L^{21E} is not a bond;
L²² is -L^{22A}-L^{22B}-L^{22C}-L^{22D}-L^{22E}-, and at least one of L^{22A}, L^{22B}, L^{22C}, L^{22D}, and L^{22E} is not a bond;
L²³ is -L^{23A}-L^{23B}-L^{23C}-L^{23D}-L^{23E}-, and at least one of L^{23A}, L^{23B}, L^{23C}, L^{23D}, and L^{23E} is not a bond; and
R² is hydrogen, halogen, —CX²₃, —CHX²₂, —CH₂X², —SOₘ₂R^{2D}, —SOᵥ₂NR^{2A}R^{2B}, NHNR^{2A}R^{2B}, —ONR^{2A}R^{2B}, —NHC═(O)NHNR^{2A}R^{2B}, —NHC(O)NR^{2A}R^{2B}, —NR^{2A}R^{2B}, C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
U³ has a structure of Formula (C),

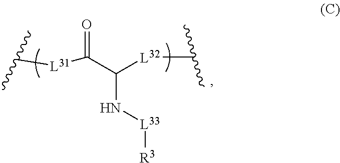

wherein:
L³¹ is -L^{31A}-L^{31B}-L^{31C}-L^{31D}-L^{31E}, and at least one of L^{31A}, L^{31B}, L^{31C}, L^{31D}, and L^{31E} is not a bond;
L³² is -L^{32A}-L^{32B}-L^{32C}-L^{32D}-L^{32E}-, and at least one of L^{32A}, L^{32B}, L^{32C}, L^{32D}, and L^{32E} is not a bond;
L³³ is -L^{33A}-L^{33B}-L^{33C}-L^{33D}-L^{33E}-, and at least one of L^{33A}, L^{33B}, L^{33C}, L^{33D}, and L^{33E} is not a bond; and
R³ is hydrogen, halogen, —CX³₃, —CHX³₂, —CH₂X³, —SOₙ₃R^{3D}, —SOᵥ₃NR^{3A}R^{3B}, —NHNR^{3A}R^{3B}, —ONR^{3A}R^{3B}, —NHC═(O)NHNR^{3A}R^{3B}, —NHC(O)NR^{3A}R^{3B}, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

Each L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B}, L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene;
q is an integer from 1 to 5;
r is an integer from 1 to 250;
s is an integer from 1 to 5;
t is an integer from 1 to 10;
z is an integer from 1 to 10;
each R^{1A}, R^{1B}, R^{1C}, R^{1D}, R^{2A}, R^{2B}, R^{2C}, R^{2D}, R^{3A}, R^{3B}, R^{3C}, R^{3D}, R^{10A}, R^{10B}, R^{10C}, and R^{10D}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} bonded to the same nitrogen atom may optionally be joined to form a substituted or unsubstituted heterocycloalkyl or substituted or unsubstituted heteroaryl; R^{2A} and R^{2B} bonded to the same nitrogen atom may optionally be joined to form a substituted or unsubstituted heterocycloalkyl or substituted or unsubstituted heteroaryl; R^{3A} and R^{3B} bonded to the same nitrogen atom may optionally be joined to form a substituted or unsubstituted heterocycloalkyl or substituted or unsubstituted heteroaryl; R^{10A} and R^{10B} bonded to the same nitrogen atom may optionally be joined to form a substituted or unsubstituted heterocycloalkyl or substituted or unsubstituted heteroaryl;
each X, X¹, X², X³, and X¹⁰ is independently —F, —Cl, —Br, or —I;
each n1, n2, n3, and n10 is independently an integer from 0 to 4; and
each v1, v2, v3, and v10 is independently 1 or 2,
provided that at least one of L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B}, L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is —S—, or —S—S—.

Also provided are, in various embodiments, a method of treating a subject suffering or susceptible to a disease or disorder and a method of treating a subject suffering or susceptible to an ocular disease or disorder. The methods include administering to the subject the pharmaceutical composition as described herein.

Further provided is a medical device including the pharmaceutical composition as described herein.

Further provided are novel compounds that may be useful for the methods described herein.

Provided herein is a monomer having a structure of Formula (X),

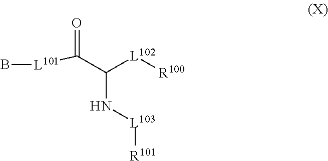

or an isomer, metabolite, prodrug, hydrate, or a salt thereof,
   wherein:
   B is a substituted or unsubstituted alkyl, substituted or unsubstituted cycloalkyl, substituted or unsubstituted heteroalkyl, substituted or unsubstituted aryl, or a substituted or unsubstituted heteroaryl,
   L¹⁰¹ is -L^{101A}-L^{101B}-L^{101C}-L^{101D}-L^{101E}-, and at least one of L^{101A}, L^{101B}, L^{101C}, L^{101D} and L^{101E} is not a bond;
   L¹⁰² is -L^{102A}-L^{102B}-L^{102C}-L^{102D}-L^{102E}- and at least one of L^{102A}, L^{102B}, L^{102C}, L^{102D}, and L^{102E} is not a bond;
   L¹⁰³ is -L^{103A}-L^{103B}-L^{103C}-L^{103D}-L^{103E}- and at least one of L^{103A}, L^{103B}, L^{103C}, L^{103D}, and L^{103E} is not a bond;
   R¹⁰⁰ is hydrogen, halogen, —CX¹⁰⁰₃, —CHX¹⁰⁰₂, —CH₂X¹⁰⁰, —SOₙ₁₀₀R^{100D}, —SOᵥ₁₀₀NR^{100A}R^{100B}, —NHNR^{100A}R^{100B}, —ONR^{100A}R^{100B}, —NHC═(O)NHNR^{100A}R^{100B}, —NHC(O)NR^{100A}R^{100B}, —NR^{100A}R^{100B}, —C(O)R^{100C}, —C(O)—OR^{100C}, —C(O)NR^{100A}R^{100B}, —OR^{100D}, —NR^{100A}SO₂R^{100D}, —NR^{100A}C(O)R^{100C}, —NR^{100A}C(O)OR^{100C}, —NR^{100A}OR^{100C}, —OCX¹⁰⁰₃, —OCHX¹⁰⁰₂, —OCH₂X¹⁰⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
   R¹⁰¹ is hydrogen, halogen, —CX¹⁰¹₃, —CHX¹⁰¹₂, —CH₂X¹⁰¹, —SOₙ₁₀₁R^{101D}, —SOᵥ₁₀₁NR^{101A}R^{101B}, —NHNR^{101A}R^{101B}, —ONR^{101A}R^{101B}, —NHC═(O)NHNR^{101A}R^{101B}, —NHC(O)NR^{101A}R^{101B}, —NR^{101A}R^{101B}, —C(O)R^{101C}, —C(O)—OR^{101C}, —C(O)NR^{101A}R^{101B}, —OR^{101D}, —NR^{101A}SO₂R^{101D}, —NR^{101A}C(O)R^{101C}, —NR^{101A}C(O)OR^{101C}, —NR^{101A}OR^{101C}, —OCX¹⁰¹₃, —OCHX¹⁰¹₂, —OCH₂X¹⁰¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
Each L^{101A}, L^{101B}, L^{101C}, L^{101D}, L^{101E}, L^{102A}, L^{102B}, L^{102C}, L^{102D}, L^{102E}, L^{103A}, L^{103B}, L^{103C}, L^{103D}, and L^{103E} independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene;
Each q100 is independently an integer from 1 to 5;
r100 is an integer from 1 to 250;
s100 is an integer from 1 to 5;
t100 is an integer from 1 to 10;
z100 is an integer from 1 to 10;
each R^{100A}, R^{100B}, R^{100C}, R^{100D}, R^{101A}, R^{101B}, R^{101C}, and R^{101D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{100A} and R^{100B} bonded to the same nitrogen atom may optionally be joined to form a substituted or unsubstituted heterocycloalkyl or substituted or unsubstituted heteroaryl;
each X, X¹⁰⁰ and X¹⁰¹ is independently —F, —Cl, —Br, or —I;
each n100 and n101 is independently an integer from 0 to 4; and
each v100, and v101 is independently 1 or 2,
   provided that at least one of L^{101A}, L^{101B}, L^{101C}, L^{101D}, L^{101E}, L^{102A}, L^{102B}, L^{102C}, L^{102D}, L^{102E}, L^{103A}, L^{103B}, L^{103C}, L^{103D}, and L^{103E} is —S—, or —S—S—.

Provided also herein is a polymer having the Formula (I) described above.

Exemplary polymers include the following structures:

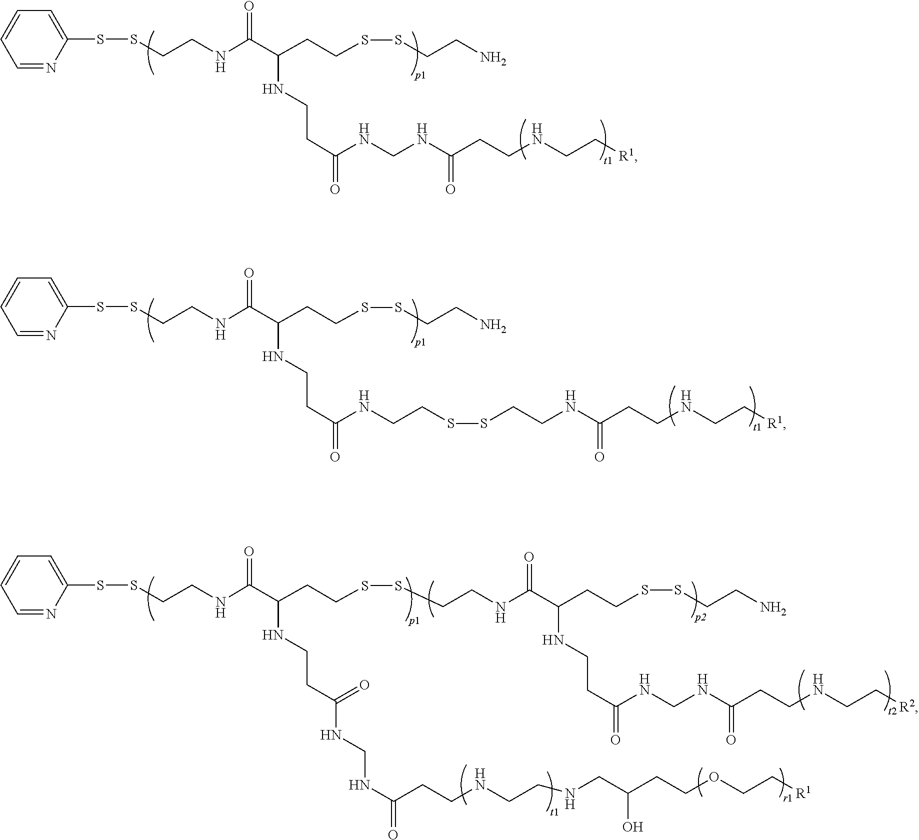

or an isomer, metabolite, prodrug, hydrate, or a salt thereof.

In a further aspects are provided for treating a subject suffering or susceptible to a disease or disorder that comprises administering to the subject a pharmaceutical composition as disclosed herein, including a composition that comprises one or more PDSA polymers or a polymer comprise a structure of any one of Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein.

In a particular aspect, methods are provided for treating a subject suffering or susceptible to an ocular disease or disorder, comprising administering to the subject a pharmaceutical composition as disclosed herein, including a composition that comprises one or more PDSA polymers or a polymer comprise a structure of any one of Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein.

In another particular aspect, methods are provided for treating a subject having erectile dysfunction, comprising administering to the subject a pharmaceutical composition as disclosed herein, including a composition that comprises one or more PDSA polymers or a polymer comprise a structure of any one of Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein.

Other aspects of the inventions are disclosed infra.

### BRIEF DESCRIPTION OF DRAWINGS

FIGS. 1A-1C: PDSA and PEGylated PDSA (PEG-PDSA) provide efficient compaction of plasmid DNA (pDNA). FIG. 1A shows DNA agarose gel migration images demonstrating the compaction of pDNA by PDSA and FIG. 1B shows DNA agarose gel migration images demonstrating the compaction of pDNA by PEG-PDSA polymers at incrementing polymer-to-pDNA weight ratios. L: 10 kb DNA ladder. FIG. 1C shows representative transmission electron micrograph of PEG-PDSA NPs. Scale bar=100 nm.

FIG. 2: pDNA payloads compacted in PDSA NPs are released preferentially in a model intracellular reducing environment over time. DNA agarose gel migration image demonstrating the release of pDNA payloads from PEI, PBAE or PDSA NPs after 1- and 3-hour incubation in 5 mM glutathione at 37 °C. L: 10 kb DNA ladder.

FIGS. 3A-3D: PDSA NPs provides greater in vitro reporter transgene expression compared to other leading formulations in ARPE-19 cells without incurring cytotoxicity. FIG. 3A shows transfection efficiency in ARPE-19 cells treated with various formulations determined by homogenate-based luciferase assay. FIG. 3B shows representative confocal images of ARPE-19 cells received Lipofectamine™3000 or PDSA NPs carrying ZsGreen plasmids. Scale bar=200 μm. FIG. 3C shows flow cytometric analysis of ARPE-19 cells treated with various formulations carrying ZsGreen plasmids. FIG. 3D shows cell viability of ARPE-19 cells treated with various formulations. **p<0.01, ***p<0.001, ****p<0.0001.

FIGS. 4A-4F: PEG-PDSA NPs efficiently penetrate vitreous gel ex vivo and provide robust in vivo reporter transgene expression in mouse retina following intravitreal administration. FIG. 4A shows representative TEM image of PEG-PDSA NPs. Scale bar=100 nm. FIG. 4B shows representative trajectories of PDSA NPs and PEG-PDSA NPs in rabbit vitreous gel. Scale bar=10 μm. FIG. 4C shows mean square displacement (MSD) at a time scale (t) of 1 second. The MSD is a square of distance traveled by an individual NP within a given time interval (i.e., time scale) and thus is directly proportional to the NP diffusion rate. FIG. 4D shows representative IVIS images of a C57BL/6Jmouse eye intravitreally treated with PEG-PDSA NPs, in comparison to a saline-treated eye. FIG. 4E shows In vivo transfection efficiency determined by tissue homogenate-based luciferase assay. FIG. 4F shows representative confocal images showing ZsGreen transgene expression (green) in a C57BL/6Jmouse eye intravitreally treated with PEG-PDSA NP, in comparison to an untreated control eye. Blue staining represents cell nuclei. GCL: ganglion cell layer; INL: inner nuclear layer; ONL: outer nuclear layer. Scale bar=20 μm. ****p<0.0001.

FIG. 5: Chemical structures and side chain functional groups of exemplary PDSA polymer variants. PDSA polymer variants will be synthesized by functionalizing the polymer backbone with various amine species (Rx: x=1-7) via reducing (i.e., disulfide) and non-reducing linkages at varying ratios. Of note, the prototype PDSA NPs tested in our pilot studies are based on a side chain R₀ and n=0 (i.e., no reducible linker for the functionalization).

FIG. 6A-6B: PEG-PDSA/pDNA NPs, unlike PDSA/pDNA NPs, provide efficient penetration through the inner limiting membrane (ILM) and transgene expression in the retinal layer of bovine vitreoretinal (VR) explants. Representative vertical images of bovine VR explants treated with PDSA/pDNA or PEG-PDSA/pDNA NPs carrying (FIG. 6A) Cy5-labeled pDNA or (FIG. 6B) ZsGreen1-endocing pDNA.

FIG. 7: PEG-PDSA/pDNA NPs provide more efficient penetration through the ILM and into the retinal layer compared to PDSA/pDNA NPs and LNPs (analogous to Comirnaty, Pfizer-BioNTech COVID-19 vaccine) in human VR explants. Representative vertical images of human VR explants treated with PDSA/pDNA or PEG-PDSA/pDNA NPs carrying Cy5-labeled pDNA, or LNPs carrying Cy5-labeled mRNA.

FIGS. 8A-8C: PDSA/mRNA and PEG-PDSA/mRNA NPs exhibit sub-100 nm particle hydrodynamic diameters, and PEG-PDSA/mRNA NPs exhibit near neutral ζ-potentials and excellent colloidal stability in PBS. (FIG. 8A) Hydrodynamic diameter and polydispersity index, and (FIG. 8B) ζ-potential of PDSA/mRNA and PEG-PDSA/mRNA NPs. (FIG. 8C) Changes in hydrodynamic diameters of PDSA/mRNA and PEG-PDSA/mRNA NPs in ultrapure water or PBS at room temperature over time for up to 48 hours. Mean±SD (n=3).

FIGS. 9A-9B: PDSA carrying mLuc or mSDF-1 showed dose-dependent production of luciferase or SDF-1 proteins, respectively, in rat penile tissue following intracavernous administration. (FIG. 9A) Luciferase activity and (FIG. 9B) SDF-1 expression of rat cavernous tissue treated with freshly prepared PDSA-R0 NPs equivalent to 10, 50, or 100 μg of 5moU-modified mLuc or mSDF-1. Mean±SD (n≥3), n.s.: no significance, **p<0.01, ***p<0.001.

FIGS. 10A-10B: PDSA/mRNA and PEG-PDSA/mRNA NPs provide efficient reporter mRNA expression in rat penile cavernous tissue comparable to DLin-MC3-DMA (MC3)-based LNP (analogous to Onpattro®) and exhibit excellent in vivo safety profiles. (FIG. 10A) Luciferase activity in rat cavernous tissue treated with PDSA-R0 NP, PEG-PDSA-R0 NP, or MC3 LNP equivalent to 50 μg of 5moU-modified mLuc. (FIG. 10B) Representative H&E images of rat cavernous tissue treated with saline, PDSA-R0 NP, or PEG-PDSA-R0 NP. Mean±SD (n≥3), n.s.: no significance, ***p<0.001.

FIGS. 11A-11B: PDSA NP variants exhibit comparable or greater in vitro mRNA expression compared to PBAE NP variants and Lipofectamine MessengerMax (Lipo) without significant cytotoxicity. (FIG. 11A) Luciferase activity and (FIG. 11B) relative cell viability of HUVECs treated with Lipo, PBAE-Rx, or PDSA-Rx carrying 50 ng of 5moU-modified mLuc. Mean±SD (n=3 for luciferase activity and n=4 for cell viability), n.s.: no significance, **p<0.01, ***p<0.001, ****p<0.0001.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl (“Me”), ethyl (“Et”), n-propyl (“Pr”), isopropyl (“iPr”), n-butyl (“Bu”), t-butyl (“t-Bu”), isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH—CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,101-dihydro-5H-dibenzo[b,f]azepin-5-yl, 1,2,3,4-tetrahydropyrido[4,3-g]isoquinolin-2-yl, 102H-benzo[b]phenoxazin-102-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The symbol “ [symbol] ” denotes the point of attachment of a chemical moiety to the remainder of a molecule or chemical formula.

The term “reducible” as used herein refers to a tendency at least in a part of a molecule or compound, by adding electrons, losing oxygen therefrom, or by adding proton. Preferably, a group of reducible moiety, under a certain condition (e.g., physiological condition, ionic strength, or acidic or basic condition), may change by adding one or more electrons, losing one or more oxygen atom therefrom, or by adding proton. For example, a disulfide group (or a bond) is reducible under a physiological condition by breaking the —S—S— bond and accepting electron and changed into —SH. The term “bioreducible” as used herein specifically refers to a tendency at least in a part of a molecule or compound, by adding electrons, losing oxygen therefrom, or by adding proton under a physiological condition (e.g., intracellular or extracellular condition).

Unless otherwise indicated, all numbers, values, and/or expressions referring to quantities of ingredients, reaction conditions, polymer compositions, and formulations used herein are to be understood as modified in all instances by the term “about” as such numbers are inherently approximations that are reflective of, among other things, the various uncertainties of measurement encountered in obtaining such values. The term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A term “nanoparticle” or “nanoparticles” as used herein refers to a particular or spherical substance that has a diameter of a predetermined size within a range from nanometer scale, which is measured by maximum distance of the particle. Exemplary nanoparticles preferably may have diameters ranges of about 1 to 999 nm, of about 1 to 900 nm, of about 1 to 800 nm, of about 1 to 700 nm, of about 1 to 600 nm, of about 1 to 500 nm, of about 1 to 400 nm, of about 1 to 300 nm, of about 1 to 200 nm, of about 1 to 100 nm, of about 1 to 50 nm, or about 1 to 20 nm, or 1 to 10 nm.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The term “EC₅₀” or “half maximal effective concentration” as used herein refers to the concentration of a molecule (e.g., small molecule, drug, antibody, chimeric antigen receptor or bispecific antibody) capable of inducing a response which is halfway between the baseline response and the maximum response after a specified exposure time. In embodiments, the EC₅₀ is the concentration of a molecule (e.g., small molecule, drug, antibody, chimeric antigen receptor or bispecific antibody) that produces 50% of the maximal possible effect of that molecule.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravitreal (e.g., injection), intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

### Polymers

Provided herein are bioreducible polymers, named poly(bioreducible amine) (PDSA), including a bioreducible backbone and multiple functional cationic amine-based side chains linked to the backbone, e.g., via bioreducible or non-bioreducible linkers. The PDSA polymer degrades preferentially in reducing environments (e.g., inside cells, or physiological condition), thereby promoting intracellular release of nucleic acid payloads for further processing after cellular uptake.

In certain embodiments, a fraction of the side chains in the PDSA polymers can be conjugated with poly(ethylene glycol) (PEG) polymers to endow the nucleic acid delivery nanoparticles (NPs) with colloidal stability in physiological environments and ability to overcome biological delivery barriers. For example, PDSA polymers form nucleic acid delivery NPs via electrostatic interactions, which exhibit markedly greater in vitro transfection efficiency compared to leading non-viral formulations. Importantly, PEGylated PDSA (PEG-PDSA) NPs, following intravitreal administration, are capable of efficiently penetrating the vitreous gel and mediating robust transgene expression throughout the mouse retinal layer. Our initial and primary application is delivery of plasmid DNA-based medicines to retina or other ocular compartments following localized administration (e.g., intravitreal, intracameral, suprachoroidal routes, etc.).

In an aspect, provided is a polymer having a structure of Formula (I),

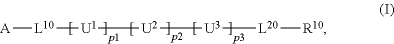

or an isomer, metabolite, prodrug, hydrate, or pharmaceutically acceptable salt thereof, wherein:
   A is a substituted or unsubstituted alkyl, substituted or unsubstituted cycloalkyl, substituted or unsubstituted heteroalkyl, substituted or unsubstituted aryl, or a substituted or unsubstituted heteroaryl,
   L¹⁰ is -L^{10A}-L^{10B}-L^{10C}-L^{10D}-L^{10E}-, and at least one of L^{10A}, L^{10B}, L^{10C}, L^{10D}, and L^{10E} is not a bond;
   L²⁰ is -L^{20A}-L^{20B}-L^{20C}-L^{20D}-L^{20E}- and at least one of L^{20A}, L^{20B}, L^{20C}, L^{20D}, and L^{20E} is not a bond;
   R¹⁰ is hydrogen, halogen, —CX¹⁰₃, —CHX¹⁰₂, —CH₂X¹⁰, —SOₙ₁₀R^{10D}, —SOᵥ₁₀NR^{10A}R^{10B}, —NHNR^{10A}R^{10B}, —ONR^{10A}R^{10B}, —NHC═(O)NHNR^{10A}R^{10B}, —NHC(O)NR^{10A}R^{10B}, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, ~OCX¹⁰₃, —OCHX¹⁰₂, —OCH₂X¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
   each p1, p2, and p3 is independently an integer from 0 to 100, and at least one of p1, p2, and p3 is not 0;
   U¹ has a structure of Formula (A),

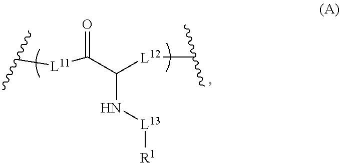

wherein:
   L¹⁰¹ is -L^{101A}-L^{101B}-L^{101C}-L^{101D}-L^{101E}-, and at least one of L^{101A}, L^{101B}, L^{101C}, L^{101D}, and L^{101E} is not a bond;
   L¹⁰² is -L^{102A}-L^{102B}-L^{102C}-L^{102D}-L^{102E}-, and at least one of L^{102A}, L^{102B}, L^{102C}, L^{102D}, and L^{102E} is not a bond;
   L¹⁰³ is -L^{103A}-L^{103B}-L^{103C}-L^{103D}-L^{103E}-, and at least one of L^{103A}, L^{103B}, L^{103C}, L^{103D}, and L^{103E} is not a bond; and
R¹ is hydrogen, halogen, —CX¹⁰³, —CHX¹⁰², —CH₂X¹, —SOₙ₁R^{1D}, —SOᵥ₁R^{1A}R^{1B}, —NHNR^{1A}R^{1B}, —ONR^{1A}R^{1B}, —NHC═(O)NHNR^{1A}R^{1B}, —NHC(O)NR^{1A}R^{1B}, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —OCX¹⁰³, —OCHX¹⁰², —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
U² has a structure of Formula (B),

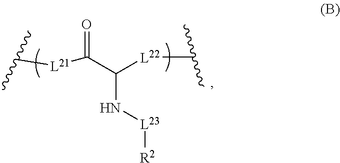

wherein:
   L²¹ is -L^{21A}-L^{21B}-L^{21C}-L^{21D}-L^{21E}-, and at least one of L^{21A}, L^{21B}, L^{21C}, L^{21D}, and L^{21E} is not a bond;
   L²² is -L^{22A}-L^{22B}-L^{22C}-L^{22D}-L^{22E}-, and at least one of L^{22A}, L^{22B}, L^{22C}, L^{22D}, and L^{22E} is not a bond;
   L²³ is -L^{23A}-L^{23B}-L^{23C}-L^{23D}-L^{23E}-, and at least one of L^{23A}, L^{23B}, L^{23C}, L^{23D}, and L^{23E} is not a bond; and
R² is hydrogen, halogen, —CX²₃, —CHX²₂, —CH₂X², —SOₙ₂R^{2D}, —SO—NRᵥ₂NR^{2A}R^{2B}, —NHNR^{2A}R^{2B}, —ONR^{2A}R^{2B}, —NHC═(O)NHNR^{2A}R^{2B}, —NHC(O)NR^{2A}R^{2B}, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
U³ has a structure of Formula (C),

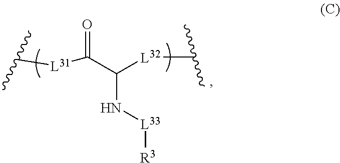

wherein:
   L³¹ is -L^{31A}-L^{31B}-L^{31C}-L^{31D}-L^{31E}-, and at least one of L^{31A}, L^{31B}, L^{31C}, L^{31D}, and L^{31E} is not a bond;
   L³² is -L^{32A}-L^{32B}-L^{32C}-L^{32D}-L^{32E}-, and at least one of L^{32A}, L^{32B}, L^{32C}, L^{32D}, and L^{32E} is not a bond;
   L³³ is -L^{33A}-L^{33B}-L^{33C}-L^{33D}-L^{33E}-, and at least one of L^{33A}, L^{33B}, L^{33C}, L^{33D}, and L^{33E} is not a bond; and
R³ is hydrogen, halogen, —CX³₃, —CHX³₂, —CH₂X³, —SOₙ₃R^{3D}, —SOᵥ₃NR^{3A}R^{3B}, —NHNR^{3A}R^{3B}, —ONR^{3A}R^{3B}, —NHC═(O)NHNR^{3A}R^{3B}, —NHC(O)NR^{3A}R^{3B}, —NR^{3A}R^{3B}, —C(O)R^{3C}, —C(O)—OR^{3C}, —C(O)R^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
Each L^{10A}, L^{10B}, L^{10C}, L^{10D}, L^{10E}, L^{101A}, L^{101B}, L^{101C}, L^{101D}, L^{101E}, L^{102A}, L^{102B}, L^{102C}, L^{102D}, L^{102E}, L^{103A}, L^{103B}, L^{103C}, L^{103D}, L^{103E}, L^{20A}, L^{20B}, L^{20C}, L^{20D}, L^{20E}, L^{21A}, L^{21B}, L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ₜ—, —(NH(CH₂)ₛ)ₜ—, —S—S—(CH₂)_{z}—, substituted or unsubstituted alkylene, or substituted or unsubstituted heteroalkylene;
q is an integer from 1 to 5;
r is an integer from 1 to 250;
s is an integer from 1 to 5;
t is an integer from 1 to 10;
z is an integer from 1 to 10;
each R^{1A}, R^{1B}, R^{1C}, R^{1D}, R^{2A}, R^{2B}, R^{2C}, R^{2D}, R^{3A}, R^{3B}, R^{3C}, R^{3D}, R^{10A}, R^{10B}, R^{10C}, and R^{10D}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} bonded to the same nitrogen atom may optionally be joined to form a substituted or unsubstituted heterocycloalkyl or substituted or unsubstituted heteroaryl; R^{2A} and R^{2B} bonded to the same nitrogen atom may optionally be joined to form a substituted or unsubstituted heterocycloalkyl or substituted or unsubstituted heteroaryl; R^{3A} and R^{3B} bonded to the same nitrogen atom may optionally be joined to form a substituted or unsubstituted heterocycloalkyl or substituted or unsubstituted heteroaryl; R^{10A} and R^{10B} bonded to the same nitrogen atom may optionally be joined to form a substituted or unsubstituted heterocycloalkyl or substituted or unsubstituted heteroaryl;
each X, X¹, X², X³, and X¹⁰ is independently —F, —Cl, —Br, or —I;
Each n1, n2, n3, and n10 is independently an integer from 0 to 4; and
Each v1, v2, v3, and v10 is independently 1 or 2,
provided that at least one of L^{10A}, L^{10B}, L^{10C}, L^{10D}, L^{10E}, L^{101A}, L^{101B}, L^{101C}, L^{101D}, L^{101E}, L^{102A}, L^{102B}, L^{102C}, L^{102D}, L^{102E}, L^{103A}, L^{103B}, L^{103C}, L^{103D}, L^{103E}, L^{20A}, L^{20B}, L^{20C}, L^{20D}, L^{20E}, L^{21A}, L^{21B}, L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is —S—, or —S—S—.

In embodiments, L¹⁰ is —S—S— or —S—S—(CH₂)_{z1}—, and z1 is an integer from 0 to 10.

In embodiments, L²⁰ is —S—S—(CH₂)_{z2}—, and z2 is an integer from 0 to 10.

In some embodiments, R¹⁰ is —NR^{10A}R^{10B}, or —OR^{10D}. Each R^{10A} and R^{10B} is independently hydrogen, substituted or unsubstituted C₁-C₄ alkyl, or 2 to 4 membered heteroalkyl, or R^{10A} and R^{10B} joined with the nitrogen attached thereto form a substituted or unsubstituted 4 to 5 membered heterocycloalkyl. R^{10D} is independently hydrogen, substituted or unsubstituted C₁-C₄ alkyl, or 2 to 4 membered heteroalkyl.

In some embodiments, R¹ is —NR^{1A}R^{1B}, or —OR^{1D}. Each R^{1A} and R^{1B} is independently hydrogen, substituted or unsubstituted C₁-C₄ alkyl, or 2 to 4 membered heteroalkyl, or R^{1A} and R^{1B} joined with the nitrogen attached thereto form a substituted or unsubstituted 4 to 5 membered heterocycloalkyl. R^{1D} is independently hydrogen, substituted or unsubstituted C₁-C₄ alkyl, or 2 to 4 membered heteroalkyl.

In embodiments, R^{1A} and R^{1B} joined with the nitrogen attached thereto form a substituted or unsubstituted 4 to 5 membered

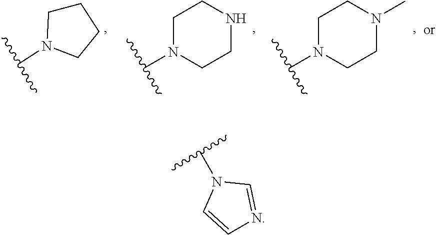

In embodiments, R¹ is —OR^{1D}. In embodiments, R^{1D} is hydrogen, substituted or unsubstituted C₁-C₄ alkyl, or 2 to 4 membered heteroalkyl. In embodiment, R^{1D} is hydrogen. In embodiment, R^{1D} is unsubstituted C₁-C₄ alkyl. In embodiment, R^{1D} is methyl. In some embodiments, R¹ is independently —NH₂,

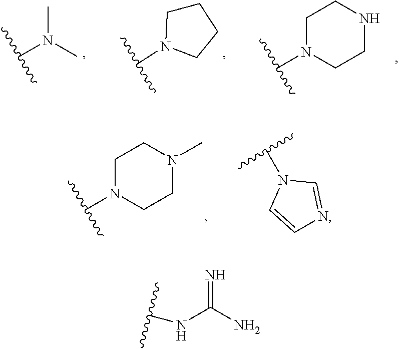

or —OH.

In some embodiments, R² is —NR^{2A}R^{2B}, or —OR^{2D}. Each R^{2A} and R^{2B} is independently hydrogen, substituted or unsubstituted C₁-C₄ alkyl, or 2 to 4 membered heteroalkyl, or R^{2A} and R^{2B} joined with the nitrogen attached thereto form a substituted or unsubstituted 4 to 5 membered heterocycloalkyl. R^{2D} is independently hydrogen, substituted or unsubstituted C₁-C₄ alkyl, or 2 to 4 membered heteroalkyl.

In embodiments, R^{2A} and R^{2B} joined with the nitrogen attached thereto form a substituted or unsubstituted 4 to 5 membered

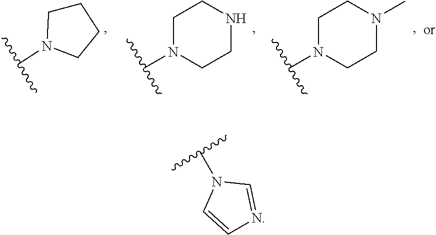

In embodiments, R² is —OR^{2D}. In embodiments, R^{2D} is hydrogen, substituted or unsubstituted C₁-C₄ alkyl, or 2 to 4 membered heteroalkyl. In embodiment, R^{2D} is hydrogen. In embodiment, R^{2D} is unsubstituted C₁-C₄ alkyl. In embodiment, R^{2D} is methyl.

In some embodiments, R² is independently —NH₂,

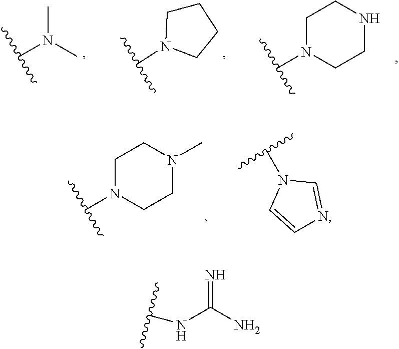

or —OH.

In some embodiments, R³ is —NR^{3A}R^{3B}, or —OR^{3D}. Each R^{3A} and R^{3B} is independently hydrogen, substituted or unsubstituted C₁-C₄ alkyl, or 2 to 4 membered heteroalkyl, or R^{3A} and R^{3B} joined with the nitrogen attached thereto form a substituted or unsubstituted 4 to 5 membered heterocycloalkyl. R^{3D} is independently hydrogen, substituted or unsubstituted C₁-C₄ alkyl, or 2 to 4 membered heteroalkyl.

In embodiments, R^{3A} and R^{3B} joined with the nitrogen attached thereto form a substituted or unsubstituted 4 to 5 membered

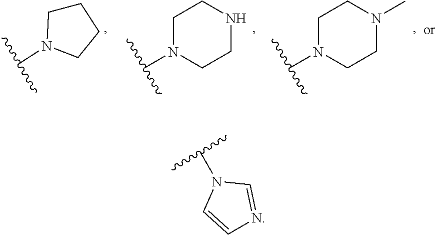

In embodiments, R³ is —OR^{3D}. In embodiments, R^{3D} is hydrogen, substituted or unsubstituted C₁-C₄ alkyl, or 2 to 4 membered heteroalkyl. In embodiment, R^{3D} is hydrogen. In embodiment, R^{3D} is unsubstituted C₁-C₄ alkyl. In embodiment, R^{3D} is methyl. In some embodiments, R³ is independently —NH₂,

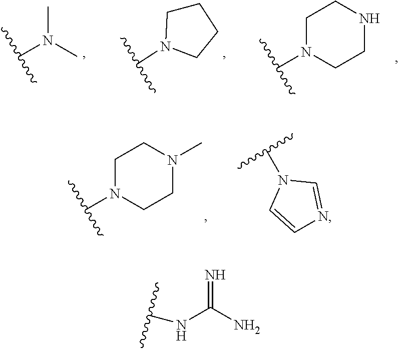

or —OH.

In embodiments, A is substituted or unsubstituted phenyl or substituted or unsubstituted pyridyl. In embodiments, A is substituted or unsubstituted phenyl. In embodiments, A is unsubstituted phenyl. In embodiments, A is substituted phenyl. In embodiments, A is substituted or unsubstituted pyridyl. In embodiments, A is unsubstituted pyridyl. In embodiments, A is substituted pyridyl.

In embodiments, the polymer has a structure of the following formula,

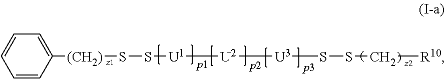

or an isomer, metabolite, prodrug, hydrate, or pharmaceutically acceptable salt thereof. U¹, U², U³, z1, z2, p1, p2, p3, and R¹⁰ are as described above. In embodiments, the polymer has a structure of the following formula,

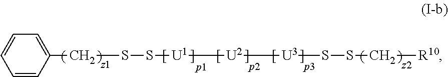

or an isomer, metabolite, prodrug, hydrate, or pharmaceutically acceptable salt thereof. U¹, U², U³, z1, z2, p1, p2, p3, and R¹⁰ are as described above.

In embodiments, the polymer has a structure of the following formula,

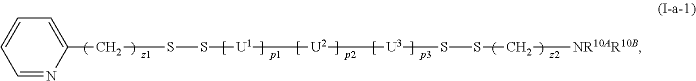

or an isomer, metabolite, prodrug, hydrate, or pharmaceutically acceptable salt thereof. U¹, U², U³, z1, z2, p1, p2, p3, R^{10A} and R^{10B} are as described above.

In embodiments, the polymer has a structure of the following formula,

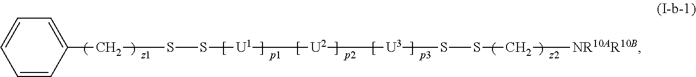

or an isomer, metabolite, prodrug, hydrate, or pharmaceutically acceptable salt thereof. U¹, U², U³, z1, z2, p1, p2, p3, R^{10A} and R^{10B} are as described above.

In embodiments, the polymer has a structure of the following formula,

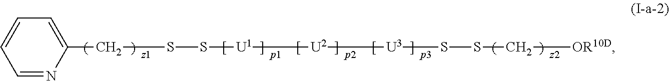

or an isomer, metabolite, prodrug, hydrate, or pharmaceutically acceptable salt thereof. U¹, U², U³, z1, z2, p1, p2, p3, and R^{10D} are as described above.

In embodiments, the polymer has a structure of the following formula,

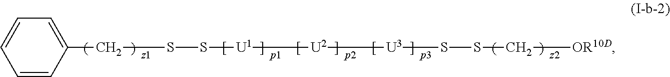

or an isomer, metabolite, prodrug, hydrate, or pharmaceutically acceptable salt thereof. U¹, U², U³, z1, z2, p1, p2, p3, and R^{10D} are as described above.

In embodiments, the polymer has a structure of Formula (II),

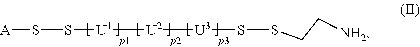

or an isomer, metabolite, prodrug, hydrate, or pharmaceutically acceptable salt thereof. A, U¹, U², U³, p1, p2, and p3 are as described above.

In embodiments, the polymer has a structure of Formula (II-a),

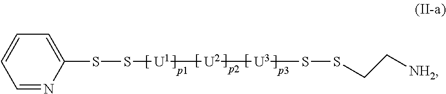

or an isomer, metabolite, prodrug, hydrate, or pharmaceutically acceptable salt thereof. U¹, U², U³, p1, p2, and p3 are as described above.

In embodiments, the polymer has a structure of Formula (II-b),

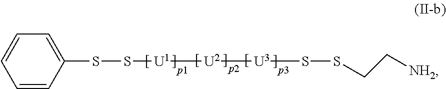

or an isomer, metabolite, prodrug, hydrate, or pharmaceutically acceptable salt thereof. U¹, U², U³, p1, p2, and p3 are as described above.

In embodiments, at least one of L¹⁰¹, L¹⁰², and L¹⁰³ includes —S—S—. In embodiments, L¹⁰¹ includes —S—S—. In embodiments, L¹⁰² includes —S—S—. In embodiments, L¹⁰³ includes —S—S—.

In certain embodiments, p2 is 0. In certain embodiments, p3 is 0. In certain embodiments, p2 and p3 are 0.

In embodiments, the polymer has a structure of the following formula (III):

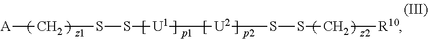

or an isomer, metabolite, prodrug, hydrate, or pharmaceutically acceptable salt thereof. A, U¹, U², p1, p2, z1, z2, and R¹⁰ are as described above.

In embodiments, the polymer has a structure of the following formula:

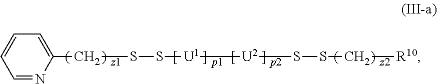

or an isomer, metabolite, prodrug, hydrate, or pharmaceutically acceptable salt thereof. U¹, U², p1, p2, z1, z2, and R¹⁰ are as described above.

In embodiments, the polymer has a structure of the following formula,

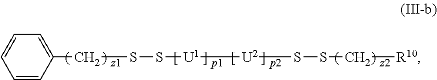

or an isomer, metabolite, prodrug, hydrate, or pharmaceutically acceptable salt thereof. U¹, U², p1, p2, z1, z2, and R¹⁰ are as described above.

In embodiments, the polymer has a structure of the following formula (IV):

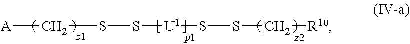

or an isomer, metabolite, prodrug, hydrate, or pharmaceutically acceptable salt thereof. A, U¹, p1, p2, z1, z2, and R¹⁰ are as described above.

In embodiments, the polymer has a structure of the following formula:

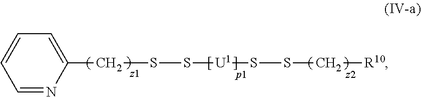

or an isomer, metabolite, prodrug, hydrate, or pharmaceutically acceptable salt thereof. U¹, p1, p2, z1, z2, and R¹⁰ are as described above.

In embodiments, the polymer has a structure of the following formula,

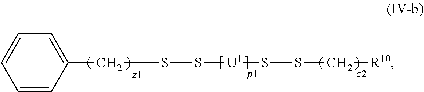

or an isomer, metabolite, prodrug, hydrate, or pharmaceutically acceptable salt thereof. U¹, U², p1, p2, z1, z2, and R¹⁰ are as described above.

In embodiments, at least one of L¹⁰¹, L¹⁰², and L¹⁰³ includes —S—S— or —S—S—(CH₂)_{z}—. In embodiments, L¹⁰¹ includes —S—S—. In embodiments, L¹⁰¹ includes —S—S—(CH₂)_{z}—. In embodiments, L¹⁰² includes —S—S—. In embodiments, L¹⁰² includes —S—S—(CH₂)_{z}—. In embodiments, L¹⁰³ includes —S—S—. In embodiments, L¹⁰³ includes —S—S—(CH₂)_{z}—. In embodiments, z is 1. In embodiments, z is 2. In embodiments, z is 3. In embodiments, z is 4. In embodiments, z is 5.

In embodiments, at least one of L¹⁰¹, L¹⁰², and L¹⁰³ includes —NH—, or —(NH(CH₂)ₛ)ₜ—. In embodiments, L¹⁰¹ includes —NH—. In embodiments, L¹⁰¹ includes —(NH(CH₂)ₛ)ₜ—. In embodiments, L¹⁰² includes —NH—. In embodiments, L¹⁰² includes —(NH(CH₂)ₛ)ₜ—. In embodiments, L¹⁰³ includes —NH—. In embodiments, L¹⁰³ includes —(NH(CH₂)ₛ)ₜ—. In embodiments, s is 1. In embodiments, s is 2. In embodiments, s is 3. In embodiments, s is 4. In embodiments, s is 5. In embodiments, t is 1. In embodiments, t is 2.

In embodiments, L^{101A}, L^{101B}, L^{101C}, L^{101D}, and L^{101E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ, —(NH(CH₂)ₛ)ₜ—, —S—S—(CH₂)_{z}—, substituted or unsubstituted alkylene, or substituted or unsubstituted heteroalkylene. In embodiments, L^{101A} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ, —(NH(CH₂)ₛ)ₜ—, —S—S—(CH₂)_{z}—, substituted or unsubstituted alkylene, or substituted or unsubstituted heteroalkylene. In embodiments, L^{101B} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments. L^{101C} is independently a bond, ~C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ, —(NH(CH₂)ₛ)ₜ—, —S—S—(CH₂)_{z}—, substituted or unsubstituted alkylene, or substituted or unsubstituted heteroalkylene. In embodiments, L^{101D} is independently a bond, —C(O)NH—, —NHC(O)—, —N(H)—, —O—, —S—, —S—S—, —C(O)—, —NHC(O)NH—, —C(O)O—, —OC(O)—, —(CH₂)_{q})ᵣ—, —(NH(CH₂)ₛ)ₜ—, —S—S—(CH₂)_{z}, substituted or unsubstituted alkylene, or substituted or unsubstituted heteroalkylene. In embodiments, L^{101E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ, —(NH(CH₂)ₛ)ₜ—, —S—S—(CH₂)_{z}—, substituted or unsubstituted alkylene, or substituted or unsubstituted heteroalkylene. In embodiments, L^{101A} is not a bond. In embodiments. L^{101B} is not a bond. In embodiments, L^{101C} is not a bond. In embodiments, 110 is not a bond. In embodiments, L^{101E} is not a bond.

In embodiments. L^{102A}, L^{102B}, L^{102C}, L^{102D}, and L^{102E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{102A} is independently a bond, ~C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{102B} is independently a bond, ~C(O)NH—, —NHC(O)—, —N(H)—O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{102C} is independently a bond, —C(O)NH—, —NHC(O)—, —N(H)—, —O—, —S—, —S—S—C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{102D} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, ~S—S—(CH₂)_{z}—, substituted or unsubstituted alkylene, or substituted or unsubstituted heteroalkylene. In embodiments, L^{102E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ, —(NH(CH₂)ₛ)ₜ—, —S—S—(CH₂)_{z}—, substituted or unsubstituted alkylene, or substituted or unsubstituted heteroalkylene. In embodiments, L^{102A} is not a bond. In embodiments. L^{102B} is not a bond. In embodiments. L^{102C} is not a bond. In embodiments, L^{102D} is not a bond. In embodiments, L^{102E} is not a bond.

In embodiments, L^{103A}, L^{103B}, L^{103C}, L^{103D}, and L^{103E} is independently a bond, —C(O)NH—, —NHC(O)—, —N(H)—, —O—, —S—, —S—S—C(O)—, —NHC(O)NH—, —C(O)O—, —OC(O)—, —(O(CH₂)_{q})ᵣ—, —(NH(CH₂)ₛ)ᵣ—, —S—S—(CH₂)_{z}—, substituted or unsubstituted alkylene, or substituted or unsubstituted heteroalkylene. In embodiments, L^{103A} is independently a bond, —C(O)NH—, —NHC(O)—, —N(H)—, —O—, —S—, —S—S—C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{103B} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{103C} is independently a bond, —C(O)NH—, —NHC(O)—, —N(H)—, —O—, —S, —S—, —S—S—, —C(O)—, —NHC(O)NH—, —C(O)O—, —OC(O)—, —(O(CH₂)_{q})^{r}, —(NH(CH₂)ₛ)ₜ—, —S—S—(CH₂)_{z}—, substituted or unsubstituted alkylene, or substituted or unsubstituted heteroalkylene. In embodiments. L^{103D} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{103E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene. In embodiments, L^{103A} is not a bond. In embodiments, L^{103B} is not a bond. In embodiments, L^{103C} is not a bond. In embodiments. L^{103D} is not a bond. In embodiments, L^{103E} is not a bond.

In embodiments, the polymer has a structure of the following formula (V):

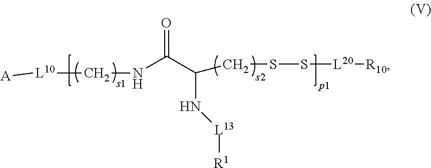

or an isomer, metabolite, prodrug, hydrate, or pharmaceutically acceptable salt thereof. A, L¹⁰, L²⁰, L¹⁰³, R¹, R¹⁰, and p1 are as described above. s1 and s2 are same as s above.

In embodiments, the polymer has a structure of the following formula:

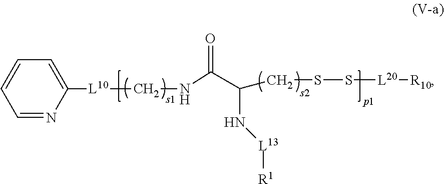

or an isomer, metabolite, prodrug, hydrate, or pharmaceutically acceptable salt thereof. L¹⁰, L²⁰, L¹⁰³, R¹, R¹⁰, and p1 are as described above. s1 and s2 are same as s above.

In embodiments, the polymer has a structure of the following formula:

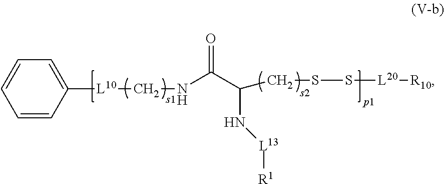

or an isomer, metabolite, prodrug, hydrate, or pharmaceutically acceptable salt thereof. L¹⁰, L²⁰, L¹⁰³, R¹, R¹⁰, p1, s1, and s2 are as described above.

In embodiments, the polymer has a structure of the following formula:

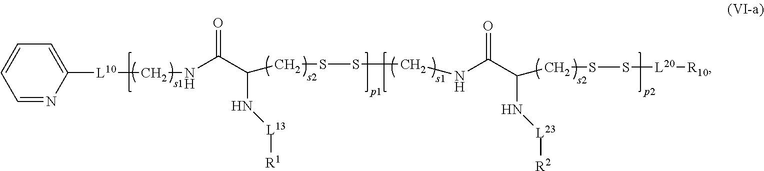

or an isomer, metabolite, prodrug, hydrate, or pharmaceutically acceptable salt thereof. L¹⁰, L²⁰, L¹⁰³, R¹, R¹⁰, p1, s1, and s2 are as described above.

In embodiments, the polymer has a structure of the following formula:

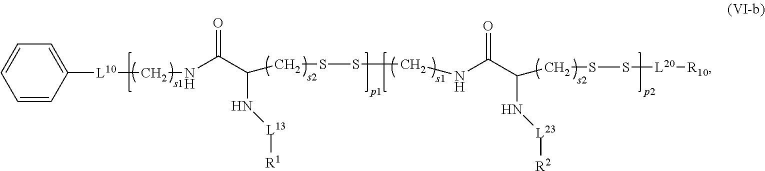

or an isomer, metabolite, prodrug, hydrate, or pharmaceutically acceptable salt thereof. L¹⁰, L²⁰, L¹⁰³, R¹, R¹⁰, p1, s1, and s2 are as described above.

In embodiments, each L¹⁰ and L²⁰ is —S—S— or substituted or unsubstituted alkylene. In embodiments, L¹⁰ is —S—S— or substituted or unsubstituted alkylene. In embodiments, L¹⁰ is —S—S—. In embodiments, L¹⁰ is substituted C₁-C₆ alkylene. In embodiments, L²⁰ is —S—S— or substituted or unsubstituted alkylene. In embodiments, L²⁰ is —S—S—. In embodiments, L²⁰ is substituted C₁-C₆ alkylene.

In embodiments, at least one of L¹⁰¹, L¹⁰², and L¹⁰³ includes —(O(CH₂)_{q})ᵣ—. In embodiments, L¹⁰¹ includes —(O(CH₂)_{q})ᵣ—. In embodiments, L¹⁰² includes —(O(CH₂)_{q})ᵣ—. In embodiments, L¹⁰³ includes —(O(CH₂)_{q})ᵣ—.

In embodiments, at least one of L²¹, L²², and L²³ includes —(O(CH₂)_{q})ᵣ—. In embodiments, L²¹ includes —(O(CH₂)_{q})ᵣ—. In embodiments, L²² includes —(O(CH₂)_{q})ᵣ—. In embodiments, L²³ includes —(O(CH)_{q})ᵣ—.

In certain embodiments, q is 2. In certain embodiments, r is an integer from 1 to 250. In certain embodiments, r is an integer from 1 to 200. In certain embodiments, r is an integer from 1 to 150. In certain embodiments, r is an integer from 1 to 100. In certain embodiments, r is an integer from 10 to 250. In certain embodiments, r is an integer from 10 to 200. In certain embodiments, r is an integer from 10 to 150. In certain embodiments, r is an integer from 10 to 100. In certain embodiments, r is an integer from 50 to 250. In certain embodiments, r is an integer from 50 to 200. In certain embodiments, r is an integer from 50 to 150. In certain embodiments, r is an integer from 50 to 100. In certain embodiments, r is an integer from 100 to 250. In certain embodiments, r is an integer from 100 to 200. In certain embodiments, r is an integer from 100 to 150.

Exemplary polymers have a structure of the following formula:

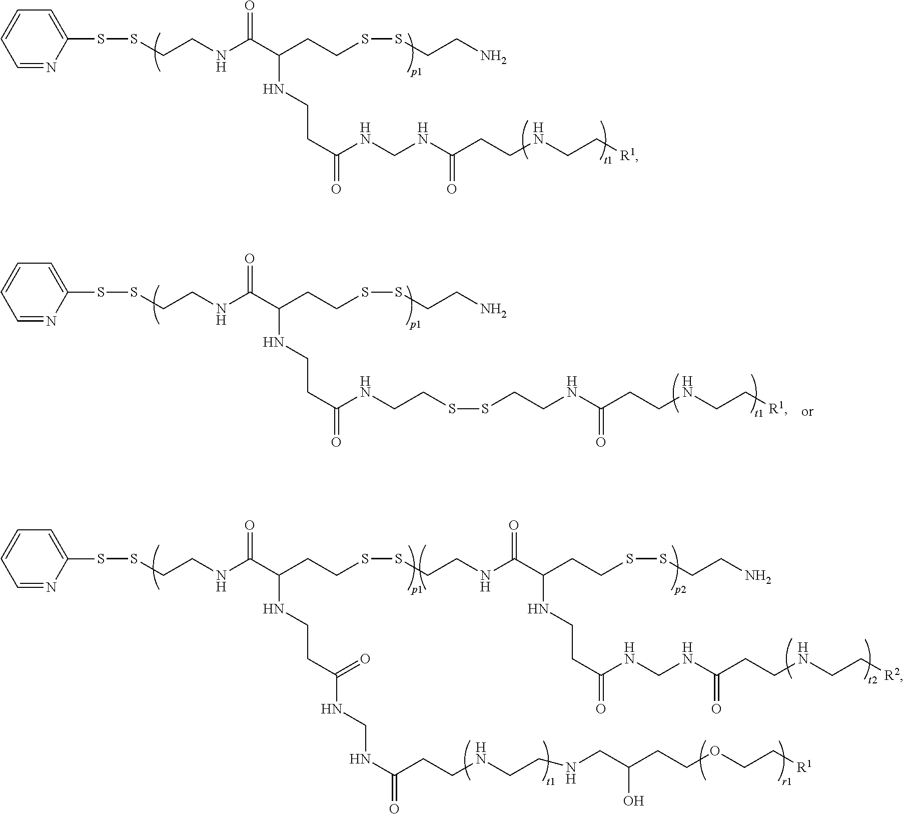

or an isomer, metabolite, prodrug, hydrate, or pharmaceutically acceptable salt thereof. p1, p2, R¹, R², t1, t2, and r1 are as described above.

In certain embodiments, each t1 and t2 is independently an integer from 1 to 10. In certain embodiments, t1 is 1. In certain embodiments, t1 is 2. In certain embodiments, t1 is 3. In certain embodiments, t1 is 4. In certain embodiments, t1 is 5. In certain embodiments, t1 is 6. In certain embodiments, t1 is 7. In certain embodiments, t1 is 7. In certain embodiments, t2 is 1. In certain embodiments, t2 is 2. In certain embodiments, t2 is 3. In certain embodiments, t2 is 4. In certain embodiments, t2 is 5. In certain embodiments, t2 is 6. In certain embodiments, t2 is 7. In certain embodiments, t2 is 8.

In certain embodiments, exemplary polymer has a structure of:

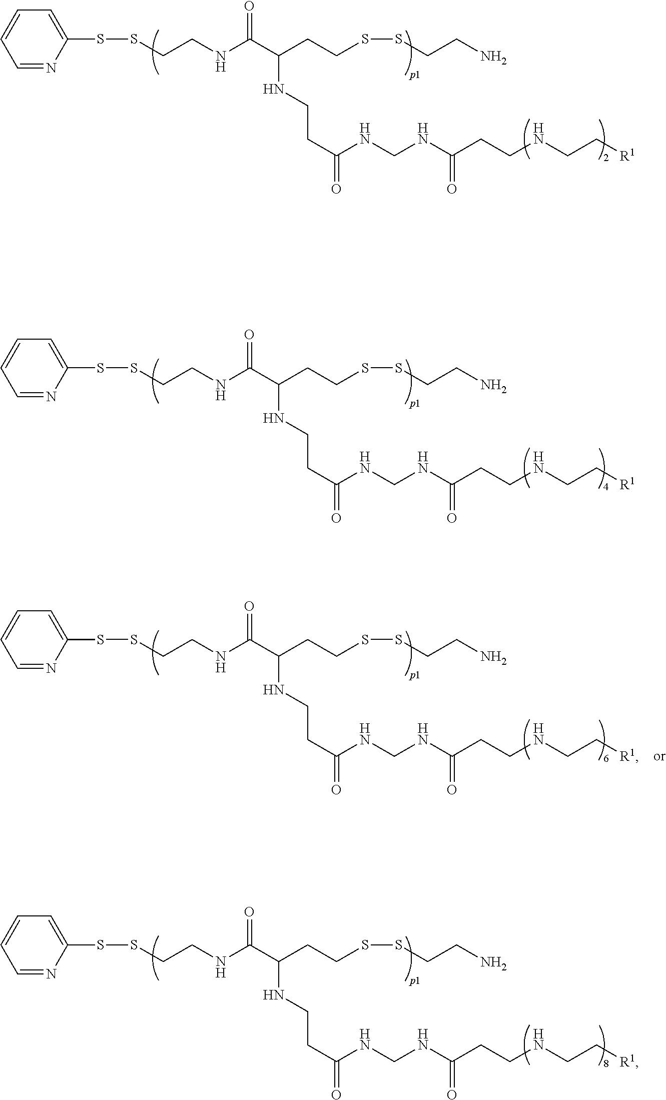

or an isomer, metabolite, prodrug, hydrate, or pharmaceutically acceptable salt thereof.

In certain embodiments, exemplary polymer has a structure of:

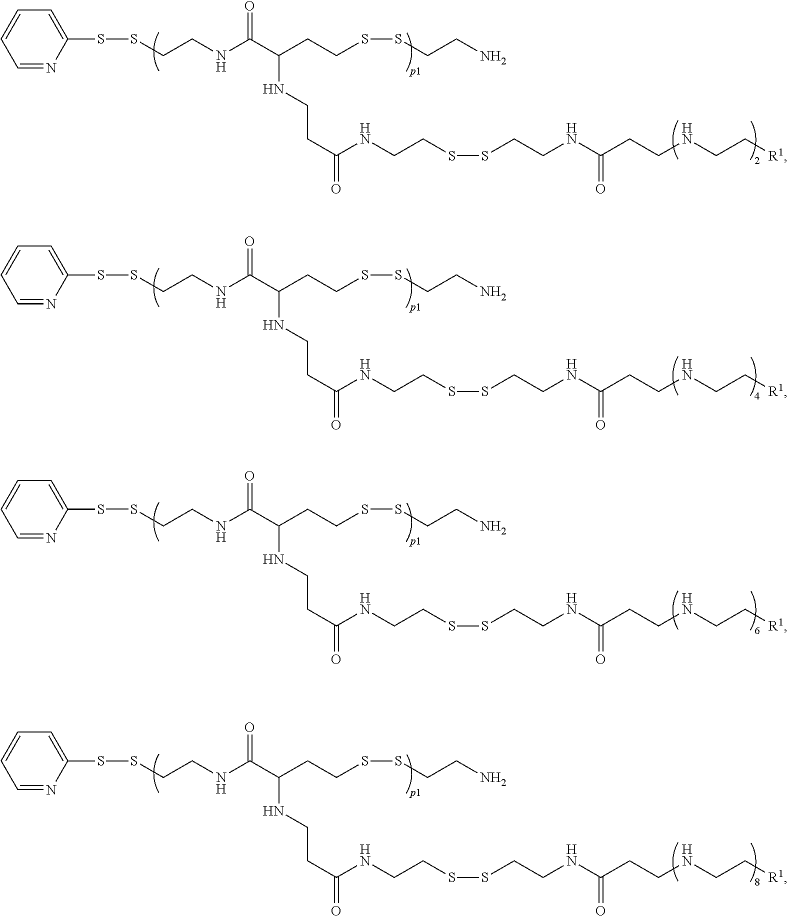

or an isomer, metabolite, prodrug, hydrate, or pharmaceutically acceptable salt thereof.

In certain embodiments, exemplary polymer has a structure of:

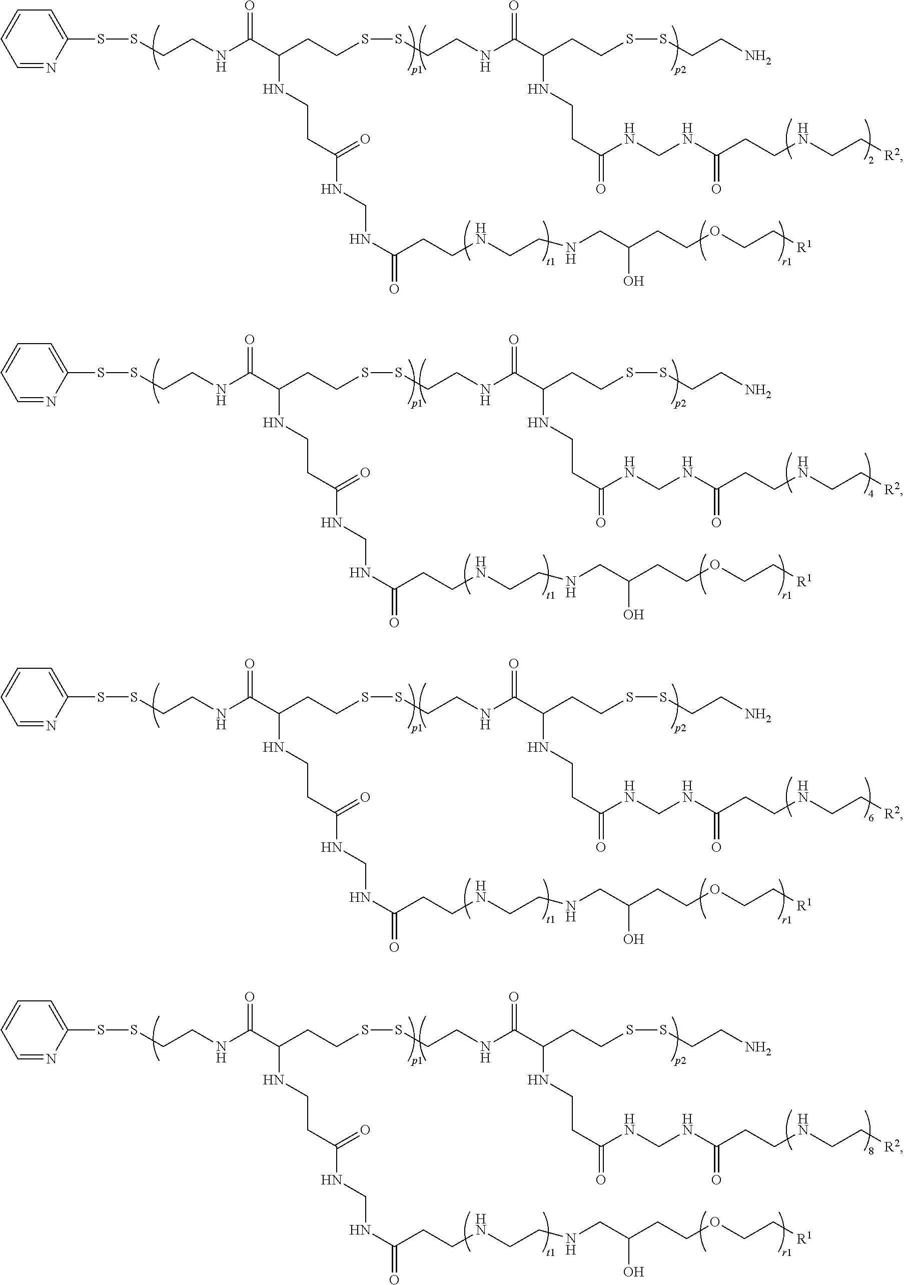

or an isomer, metabolite, prodrug, hydrate, or pharmaceutically acceptable salt thereof. In embodiments, t1 is 2, 4, 6, or 8.

In certain embodiments, exemplary polymer has a structure of:

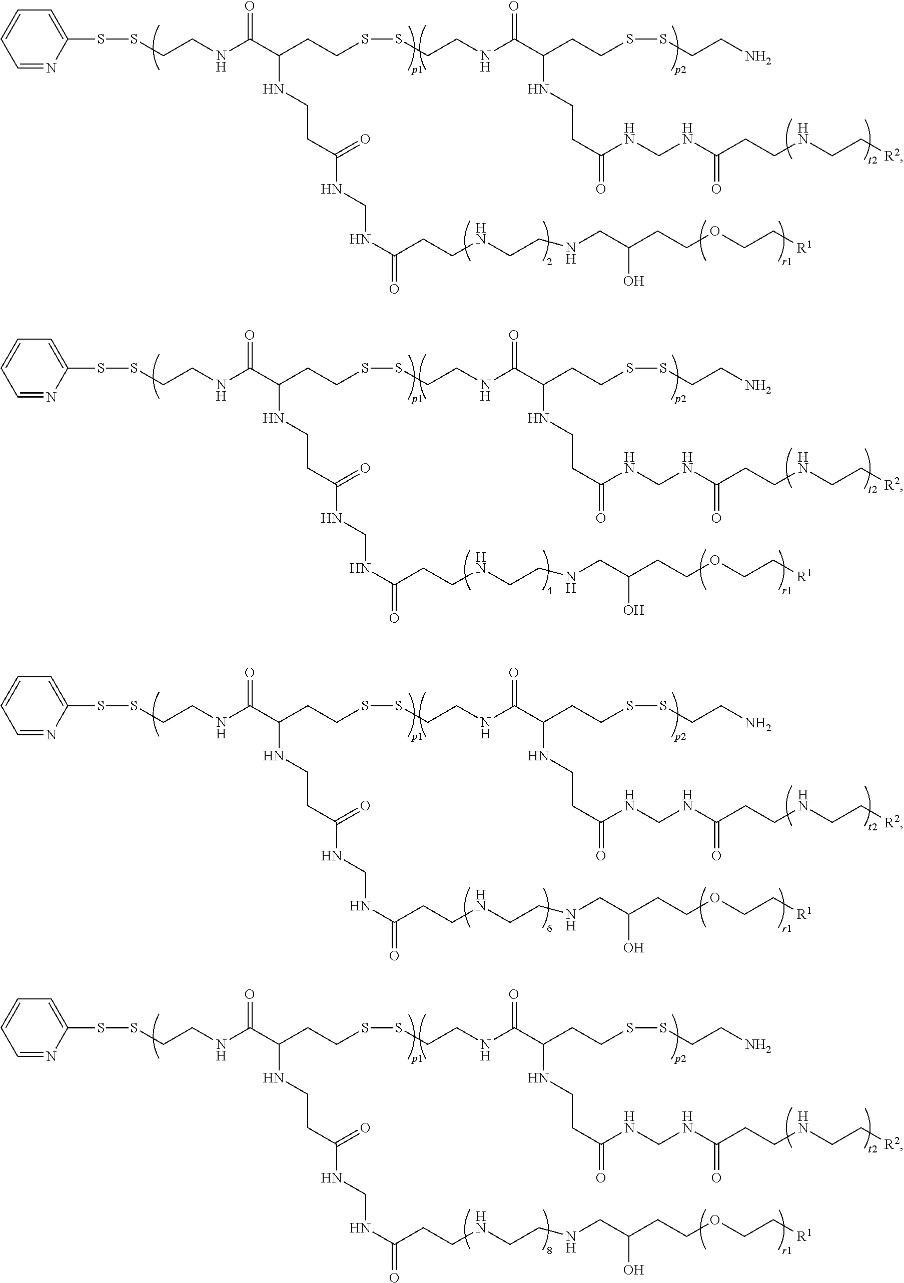

or an isomer, metabolite, prodrug, hydrate, or pharmaceutically acceptable salt thereof. In embodiments, t2 is 2, 4, 6, or 8.

In certain embodiments, r1 is an integer from 1 to 250. In certain embodiments, r1 is an integer from 1 to 200. In certain embodiments, r1 is an integer from 1 to 150. In certain embodiments, r1 is an integer from 1 to 100. In certain embodiments, r1 is an integer from 10 to 250. In certain embodiments, r1 is an integer from 10 to 200. In certain embodiments, r1 is an integer from 10 to 150. In certain embodiments, r1 is an integer from 10 to 100. In certain embodiments, r1 is an integer from 50 to 250. In certain embodiments, r1 is an integer from 50 to 200. In certain embodiments, r1 is an integer from 50 to 150. In certain embodiments, r1 is an integer from 50 to 100. In certain embodiments, r1 is an integer from 100 to 250. In certain embodiments, r1 is an integer from 100 to 200. In certain embodiments, r1 is an integer from 100 to 150.

In certain embodiments, when L¹⁰³, L²³, or L³³ includes —(O(CH₂)_{q})ᵣ—, R¹, R², or R³, respectively may be substituted or unsubstituted alkyl. In certain embodiments, when L¹⁰³, L²³, or L³³ includes —(O(CH₂)_{q})ᵣ—, R¹, R², or R³, respectively, is unsubstituted alkyl. In certain embodiments, when L¹⁰³, L²³, or L³³ includes —(O(CH₂)_{q})ᵣ—, R¹, R², or R³, respectively, is unsubstituted C₁-C₃ alkyl. In certain embodiments, when L¹⁰³, L²³, or L³³ includes —(O(CH₂)_{q})ᵣ—, R¹, R², or R³, respectively, is methyl.

### Pharmaceutical Composition

Provided herein are pharmaceutical compositions (“compositions”) including a poly(disulfide amine) polymer (PDSA polymer) as described herein. The PDSA polymer preferably has a structure of Formula (I), (I-a-1), (I-b-1), (I-a-2), (I-b-2), (II), (II-a), (II-b), (III), (III-a), (III-b), (IV), (IV-a), (IV-b), (V-a), (V-b), (VI-a), (VI-b), or its subordinates, or an isomer, metabolite, prodrug, hydrate, or pharmaceutically acceptable salt thereof.

In embodiments, the compositions include a plurality of nanoparticles. In certain embodiments, the nanoparticles include one or more kinds of the poly(disulfide amine) polymers, e.g., by forming a shell structure including the PDSA polymers.

In embodiments, the nanoparticles have a mean particle diameter of less than about 10 nm. In embodiments, the nanoparticles have a mean particle diameter of less than about 50 nm. In embodiments, the nanoparticles have a mean particle diameter of less than about 100 nm. In embodiments, the nanoparticles have a mean particle diameter of less than about 150 nm. In embodiments, the nanoparticles have a mean particle diameter of less than about 200 nm. In embodiments, the nanoparticles have a mean particle diameter of less than about 250 nm. In embodiments, the nanoparticles have a mean particle diameter of less than about 300 nm. In embodiments, the nanoparticles have a mean particle diameter of less than about 400 n.m. In embodiments, the nanoparticles have a mean particle diameter of less than about 500 n.m. In embodiments, the nanoparticles have a mean particle diameter of less than about 600 nm. In embodiments, the nanoparticles have a mean particle diameter of less than about 700 nm. In embodiments, the nanoparticles have a mean particle diameter of less than about 800 nm. In embodiments, the nanoparticles have a mean particle diameter of less than about 900 nm. In embodiments, the nanoparticles have a mean particle diameter of less than about 950 nm.

In embodiments, the composition includes a poly(alkylene glycol) (PEG) component. For example, the PDSA polymer is attached or conjugated with the poly(ethylene glycol) (PEG) polymers to endow the nucleic acid delivery nanoparticles (NPs) with colloidal stability in physiological environments and ability to overcome biological delivery barriers. In embodiments, the composition includes the PEGylated PDSA (PEG-PDSA) NPs. In embodiments, the composition includes a polyethylene glycol coating layer, e.g., formed on the nanoparticles or on polymer aggregates or layers.

In embodiments, the composition includes an anionic component. In embodiments, the anionic component may include any biological molecules (e.g., polynucleotide, oligonucleotide, proteins, antibodies, steroids, phospholipids, or etc.), synthetic or natural polymers, small molecules.

In embodiments, the composition includes a therapeutic agent. In embodiments, the therapeutic agent may include any biological molecules (e.g., nucleic acids, polynucleotide, oligonucleotide, proteins, antibodies, antigens, virus, steroids, phospholipids, or etc.), synthetic or natural polymers, small molecules.

In certain embodiments, the composition include a polynucleotide. In certain embodiments, the polynucleotide includes a single- or double-stranded DNA, a single- or double-stranded RNA, a plasmid DNA, a single- or double-stranded siRNA, an antisense oligonucleotide, a ribozyme, or a catalytic RNA or nucleotide. In certain embodiments, the composition includes nucleic acids (e.g., mRNA, siRNA, etc.). In certain embodiments, the composition includes mRNA. In certain embodiments, the composition includes siRNA. In certain embodiments, the composition includes microRNA (miRNA). In certain embodiments, the composition includes self amplifying RNA (saRNA).

In certain embodiments, the composition includes a peptide or a peptide nucleic acid. In certain embodiments, the composition includes a polynucleotide and/or a peptide or a peptide nucleic acid.

The pharmaceutical composition may be prepared and administered in a wide variety of dosage formulations. Compounds described may be administered orally, rectally, or by injection (e.g., intravitreally, intravenously, intramuscularly, intracutaneously, subcutaneously, intraduodenally, or intraperitoneally). In certain embodiments, the pharmaceutical composition is formulated for intravitreal (e.g., injection) administration.

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material. In powders, the carrier may be a finely divided solid in a mixture with the finely divided active component. In tablets, the active component may be mixed with the carrier having the necessary binding properties in suitable proportions and compacted in the shape and size desired. The powders and tablets may contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution. Aqueous solutions suitable for oral or injection use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical composition may include compositions wherein the active ingredient (e.g., nucleic acids or biological molecules) is contained in a therapeutically effective amount, i.e., in an amount effective to achieve its intended purpose. The actual amount effective for a particular application will depend, inter aim, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state. Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### USE

The disclosure provides methods of using the polymers (e.g., PDSA polymers), pharmaceutical compositions including the polymers as described herein, e.g., PDSA polymer preferably has a structure of Formula (I), (I-a-1), (I-b-1), (I-a-2), (I-b-2), (II-a), (II-b), (III-a), (III-b), (IV-a), (IV-b), (V-a), (V-b), (VI-a), (VI-b), or its subordinates, or an isomer, metabolite, prodrug, hydrate, or pharmaceutically acceptable salt thereof, or a device including the polymers or the compositions.

In an aspect, provided is a method of treating a subject suffering or susceptible to a disease or disorder. The method includes administering to the subject the pharmaceutical composition as described herein.

In an aspect, provided also is a method of treating a subject suffering or susceptible to an ocular disease or disorder. The method includes administering to the subject the pharmaceutical composition as described herein. The composition may be administered by intravitreal (e.g., injection) administration. The composition may be administered by topical administration to an eye.

A wide variety of diseases and disorders with the present compositions and can vary with the therapeutic agents delivered with the polymer material. Thus, as discussed, nucleic acid can be administered that is useful in gene therapy, for example in order to express a desired gene in a cell or group of cells. Such nucleic acid is typically is operatively linked to appropriate regulatory sequences such as promoters, enhancers and the like such that the plasmid DNA is expressed once it has been delivered to the cells to be treated.

The present compositions and methods are particularly useful to treat a variety of ocular diseases and disorders including a variety of inherited or acquired diseases including, without limitation, retinitis pigmentosa, Stargardt disease, and macular degeneration including age-related macular degeneration. In certain aspects, the compositions and methods described herein may be used to treat diseases of the inner retina (such as vascular occlusions and diabetic retinopathy), diseases of the optic nerve (such as optic neuropathies and glaucoma), diseases of the anterior portion of the eye (such as corneal endothelial deficiency, cataract, ocular hypertension, and glaucoma), inflammatory diseases of the eye (such as uveitis, ocular trauma, and ocular infections), and neoplastic diseases of the eye (such as choroidal tumors, epithelial tumors, and metastatic disease).

The present compositions and methods also may be useful for treating other diseases and disorders susceptible to gene therapy including, for example, haemophilia B (Factor IX), cystic fibrosis (CTFR) and spinal muscular atrophy (SMN-1).

In further aspect, provided also is a method of treating a subject having (or diagnosed with) erectile dysfunction. The method includes administering to the subject a pharmaceutical composition as disclosed herein. The composition may be administered by intravitreal (e.g., injection) administration.

The subject (e.g., human or human patient) may have inability to provide sufficient blood flow to the penis to fill the corpora cavernosa and achieve an erection. The subject (e.g., human or human patient) having erectile dysfunction may not have normal erection activity which is resulted from various physiological impotence (e.g., diseases or injury in nerves, blood vessels or hormones that control erectile ability, diabetes mellitus, vascular diseases, impotence following radical surgery, spinal cord injury and other traumas, other endocrine problems and multiple sclerosis, prostate infections, drug abuse, alcoholism, side effects of therapeutical medicine, and smoking) and/or psychological factors (e.g. anxiety, depression, tension and stress). In some embodiments, the methods may further include diagnosing or identifying a patient group having at least one of the physiological impotence and/or psychological factors.

Nucleic acid also can be administered that is used in immunisation to express one or more antigens against which it is desired to produce an immune response. Thus, the nucleic acid to be loaded into the exosome can encode one or more antigens against which is desired to produce an immune response, such as tumour antigens, antigens from pathogens such as viral, bacterial or fungal pathogens.

Nucleic acid also can be administered that is used in gene silencing. Such gene silencing may be useful in therapy to switch off aberrant gene expression or in animal model studies to create single or more genetic knock outs. Such nucleic acid may be provided in the form of siRNAs. For example, RNAi molecules including siRNAs can be used to knock down DMPK with multiple CUG repeats in muscle cells for treatment of myotonic dystrophy. In other examples, plasmids expressing shRNA that reduces the mutant Huntington gene (htt) responsible for Huntington's disease can be delivered with neuron specific exosomes. Other target genes include BACE-1 for the treatment of Alzheimer's disease. Some cancer genes may also be targeted with siRNA or shRNAs, such as ras, c-myc and VEGFR-2. Brain targeted siRNA loaded exosomes may be particularly useful in the silencing of BACE-1 in Alzheimer's disease, silencing of alpha-synuclein in Parkinson's disease, silencing of htt in Huntingdon's disease and silencing of neuronal caspase-3 used in the treatment of stroke to reduce ischaemic damage.

Antisense modified oligonucleotides including 2′-O-Me compounds also can be administered. For example, such oligonucleotides can be designed to induce exon-skipping for example the mutant dystrophin gene can be delivered to muscle cells for the treatment of Duchenne Muscular Dystrophy, antisense oligonucleotides which inhibit hairpin loops, for example in the treatment of myotonic dystrophy and trans-splicing oligonucleotides, for example for the treatment of spinal muscular atrophy.

In an aspect, provided is a medical device including the pharmaceutical composition as described herein. The medical device may be supplied with an applicator (e.g., surgical applicator, or injection applicator). In some embodiments, the medical device is included in a kit.

### Monomers

The disclosure also provides monomers that can be used for preparing the polymers (e.g., PDSA polymers) described herein. The monomers may constitute about 10%, about 20%, about 30%, about 40%, about 50%, about 60%, about 70%, about 80%, about 90%, about 95%, of the total monomers polymerized into the PDSA polymer. The monomers may be suitably combined with a solvent component, cross-linking agents, and other additive to form a resin or resin dispersion prior to be polymerization reaction or curing (e.g., by light or heat).

In an aspect, provided is a monomer having a structure of Formula (X),

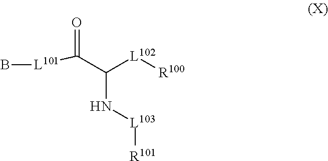

or an isomer, metabolite, prodrug, hydrate, or a salt thereof,
   wherein:
   B is a substituted or unsubstituted alkyl, substituted or unsubstituted cycloalkyl, substituted or unsubstituted heteroalkyl, substituted or unsubstituted aryl, or a substituted or unsubstituted heteroaryl,
   L¹⁰¹ is -L^{101A}-L^{101B}-L^{101C}-L^{101D}-L^{101E}-, and at least one of L^{101A}, L^{101B}, L^{101C}, L^{101D}, and L^{101E} is not a bond;
   L¹⁰² is -L^{102A}-L^{102B}-L^{102C}-L^{102D}-L^{102E}- and at least one of L^{102A}, L^{102B}, L^{102C}, L^{102D}, and L^{102E} is not a bond;
   L¹⁰³ is -L^{103A}-L^{103B}-L^{103C}-L^{103D}-L^{103E}- and at least one of L^{103A}, L^{103B}, L^{103C}, L^{103D}, and L^{103E} is not a bond;
   R¹⁰⁰ is hydrogen, halogen, —CX¹⁰⁰₃, —CHX¹⁰⁰₂, —CH₂X¹⁰⁰, —SOₙ₁₀₀R^{100D}, —SOᵥ₁₀₀NR^{100A}R^{100B}, —NHNR^{100A}R^{100B}, —ONR^{100A}R^{100B}, NHC═(O)NHNR^{100A}R^{100B}, —NHC(O)NR^{100A}R^{100B}, —NR^{100A}R^{100B}, —C(O)R^{100C}, —C(O)—OR^{100C}, —C(O)NR^{100A}R^{100B}, —OR^{100D}, —NR^{100A}C(O)R^{100C}, —NR^{100A}C(O)OR^{100C}, —NR^{100A}OR^{100C}, —OCX¹⁰⁰₃, —OCHX¹⁰⁰₂, —OCH₂X¹⁰⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
   R¹⁰¹ is hydrogen, halogen, —CX¹⁰¹₃, —CHX¹⁰¹₂, —CH₂X¹⁰¹, —SOₙ₁₀₁R^{101D}, —SOᵥ₁₀₁NR^{101A}R^{101B}, —NHNR^{101A}R^{101B}, —ONR^{101A}R^{101B}, —NHC═(O)NHNR^{101A}R^{101B}, —NHC(O)NR^{101A}R^{101B}, —NR^{101A}R^{101B}, —C(O)R^{101C}, —C(O)—OR^{101C}, —C(O)NR^{101A}R^{101B}, —OR^{101D}, —NR^{101A}SO₂R^{101D}, —NR^{101A}C(O)R^{101C}, —NR^{101A}C(O)OR^{101C}, —NR^{101A}OR^{101C}, —OCX¹⁰¹₃, —OCHX¹⁰¹₂, —OCH₂X¹⁰¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
   Each L^{101A}, L^{101B}, L^{101C}, L^{101D}, L^{101E}, L^{102A}, L^{102B}, L^{102C}, L^{102D}, L^{102E}, L^{103A}, L^{103B}, L^{103C}, L^{103D}, and L^{103E} independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)_{q100})ᵣ₁₀₀—, —S—, —S—S—(CH₂)_{z100}—, substituted or unsubstituted alkylene, or substituted or unsubstituted heteroalkylene;
   Each q100 is independently an integer from 1 to 5;
   r100 is an integer from 1 to 250;
   s100 is an integer from 1 to 5;
   t100 is an integer from 1 to 10;
   z100 is an integer from 1 to 10;
   each R^{100A}, R^{100B}, R^{100C}, R^{100D}, R^{101A}, R^{101B}, R^{101C}, and R^{101D} is independently hydrogen, —CX₃, —CN, ~COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{100A} and R^{100B} bonded to the same nitrogen atom may optionally be joined to form a substituted or unsubstituted heterocycloalkyl or substituted or unsubstituted heteroaryl;
   each X, X¹⁰⁰ and X¹⁰¹ is independently —F, —Cl, ~Br, or —I; and
   each n100 and n101 is independently an integer from 0 to 4;
   provided that at least one of L^{101A}, L^{101B}, L^{101C}, L^{101D}, L^{101E} L^{102A}, L^{102B}, L^{102C}, L^{102D}, L^{102E}, L^{103A}, L^{103B}, L^{103C}, L^{103D}, and L^{103E} is —S—, or —S—S—.

In embodiments, B is substituted or unsubstituted phenyl or substituted or unsubstituted pyridyl. In embodiments, B is substituted or unsubstituted phenyl. In embodiments, B is unsubstituted phenyl. In embodiments, B is substituted phenyl. In embodiments, B is substituted or unsubstituted pyridyl. In embodiments, B is unsubstituted pyridyl. In embodiments, B is substituted pyridyl.

In some embodiments, L^{101A} is —S—S—. In some embodiments, L^{102E} is —S—. In some embodiments, R¹⁰⁰ is hydrogen.

In some embodiments, L^{101A} is —S—S—. In some embodiments, L^{103E} is —S—. In some embodiments, R¹⁰⁰ is hydrogen.

In some embodiments, R¹⁰⁰ is —NR^{100A}R^{100B}, or —OR^{100D}. Each R^{100A} and R^{100B} is independently hydrogen, substituted or unsubstituted C₁-C₄ alkyl, or 2 to 4 membered heteroalkyl, or R^{100A} and R^{100B} joined with the nitrogen attached thereto form a substituted or unsubstituted 4 to 5 membered heterocycloalkyl. R^{100D} is independently hydrogen, substituted or unsubstituted C₁-C₄ alkyl, or 2 to 4 membered heteroalkyl.

In embodiments, R^{100A} and R^{100B} joined with the nitrogen attached thereto form a substituted or unsubstituted 4 to 5 membered

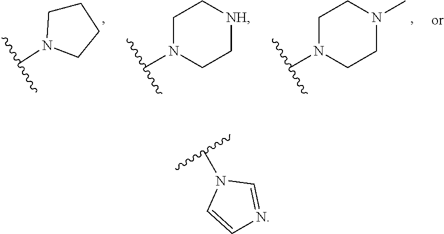

In embodiments, R¹⁰⁰ is —OR^{100D}. In embodiments, R^{100D} is hydrogen, substituted or unsubstituted C₁-C₄ alkyl, or 2 to 4 membered heteroalkyl. In embodiment, R^{100D} is hydrogen. In embodiment, R^{100D} is unsubstituted C₁-C₄ alkyl. In embodiment, R^{100D} is methyl.

In some embodiments, R¹⁰⁰ is independently —NH₂,

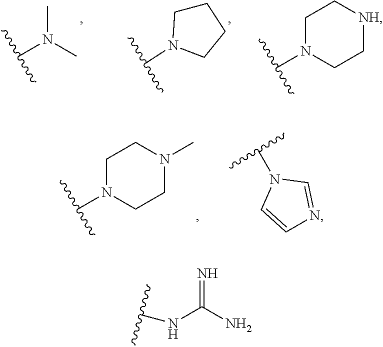

or —OH.

In embodiments, R^{101A} and R^{101B} joined with the nitrogen attached thereto form a substituted or unsubstituted 4 to 5 membered

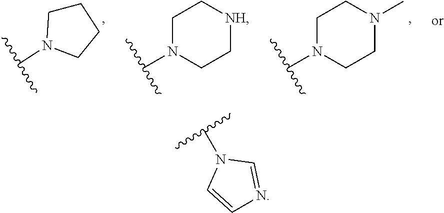

In embodiments, R¹⁰¹ is —OR^{101D}. In embodiments, R^{101D} is hydrogen, substituted or unsubstituted C₁-C₄ alkyl, or 2 to 4 membered heteroalkyl. In embodiment, R^{101D} is hydrogen. In embodiment, R^{101D} is unsubstituted C₁-C₄ alkyl. In embodiment, R^{101D} is methyl.

In some embodiments, R¹⁰¹ is independently —NH₂,

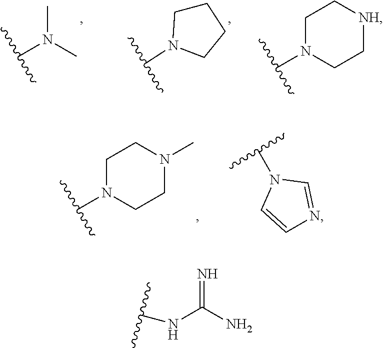

or —OH.

In some embodiments, R¹⁰¹ is independently —NH₂. In some embodiments, R¹⁰¹ is independently —OH.

In embodiments, at least one of L¹⁰¹, L¹⁰², and L¹⁰³ includes —S—S— or —S—S—(CH₂)_{z100}—. In embodiments, L¹⁰¹ includes —S—S—. In embodiments, L¹⁰¹ includes —S—S—(CH₂)_{z100}—. In embodiments, L¹⁰² includes —S—S—, In embodiments, L¹⁰² includes —S—S—(CH₂)_{z100}—. In embodiments, L¹⁰³ includes —S—S—, In embodiments, L¹⁰³ includes —S—S—(CH₂)_{z100}—. In embodiments, z100 is 1. In embodiments, z100 is 2. In embodiments, z100 is 3. In embodiments, z100 is 4. In embodiments, z100 is 5.

In embodiments, at least one of L¹⁰¹, L¹⁰², and L¹⁰³ includes —NH—, or —(NH(CH₂)ₛ)ₜ—. In embodiments, L¹⁰¹ includes —NH—. In embodiments, L¹⁰¹ includes —(NH(CH₂)ₛ)ₜ—. In embodiments, L¹⁰² includes —NH—. In embodiments, L¹⁰² includes —(NH(CH₂)ₛ)ₜ—. In embodiments, L¹⁰³ includes —NH—. In embodiments, L¹⁰³ includes —(NH(CH₂)ₛ)ₜ—. In embodiments, s is 1. In embodiments, s is 2. In embodiments, s is 3. In embodiments, s is 4. In embodiments, s is 5. In embodiments, t is 1. In embodiments, t is 2.

In embodiments, L^{101A}, L^{101B}, L^{101C}, L^{101D}, and L^{101E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101A} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101B} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101C} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101D} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ)ₜ—, —S—S—(CH₂)_{z100}—, substituted or unsubstituted alkylene, or substituted or unsubstituted heteroalkylene. In embodiments, L^{101A} is not a bond. In embodiments, L^{101B} is not a bond. In embodiments, L^{101C} is not a bond. In embodiments, L^{101D} is not a bond. In embodiments, L^{101E} is not a bond.

In embodiments, L^{102A}, L^{102B}, L^{102C}, L^{102D}, and L^{102E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{102A} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments. L^{102B} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ᵣ₁₀₀—, —S—S—(CH₂)_{z100}—, substituted or unsubstituted alkylene, or substituted or unsubstituted heteroalkylene. In embodiments, L^{102C} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, ~S—S—(CH₂)_{z100}—, substituted or unsubstituted alkylene, or substituted or unsubstituted heteroalkylene. In embodiments. L^{102C} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{102E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ₜ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{102A} is not a bond. In embodiments, L^{102B} is not a bond. In embodiments, L^{102C} is not a bond. In embodiments, L^{102D} is not a bond. In embodiments, L^{102E} is not a bond.

In embodiments, L^{103A}, L^{103B}, L^{103C}, L^{103D}, and L^{103E} is independently a bond, —C(O)NH—, —NHC(O)—, —N(H)—, —O—, —S—, —S—S—, —C(O)—, ~NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{103A} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, ~S—S—(CH₂)_{z100}—, substituted or unsubstituted alkylene, or substituted or unsubstituted heteroalkylene. In embodiments. L^{103B} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{103C} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{103D} is independently a bond, —C(O)NH—, —NHC(O)—, —N(H)—O—S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments, L^{103E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q100})ᵣ₁₀₀—, —(NH(CH₂)ₛ₁₀₀)ₜ₁₀₀—, —S—S—(CH₂)_{z100}—, substituted or unsubstituted alkylene, or substituted or unsubstituted heteroalkylene. In embodiments. L^{103A} is not a bond. In embodiments, L^{103B} is not a bond. In embodiments, L^{103C} is not a bond. In embodiments, L^{103D} is not a bond. In embodiments, L^{103E} is not a bond.

Exemplary monomers may include

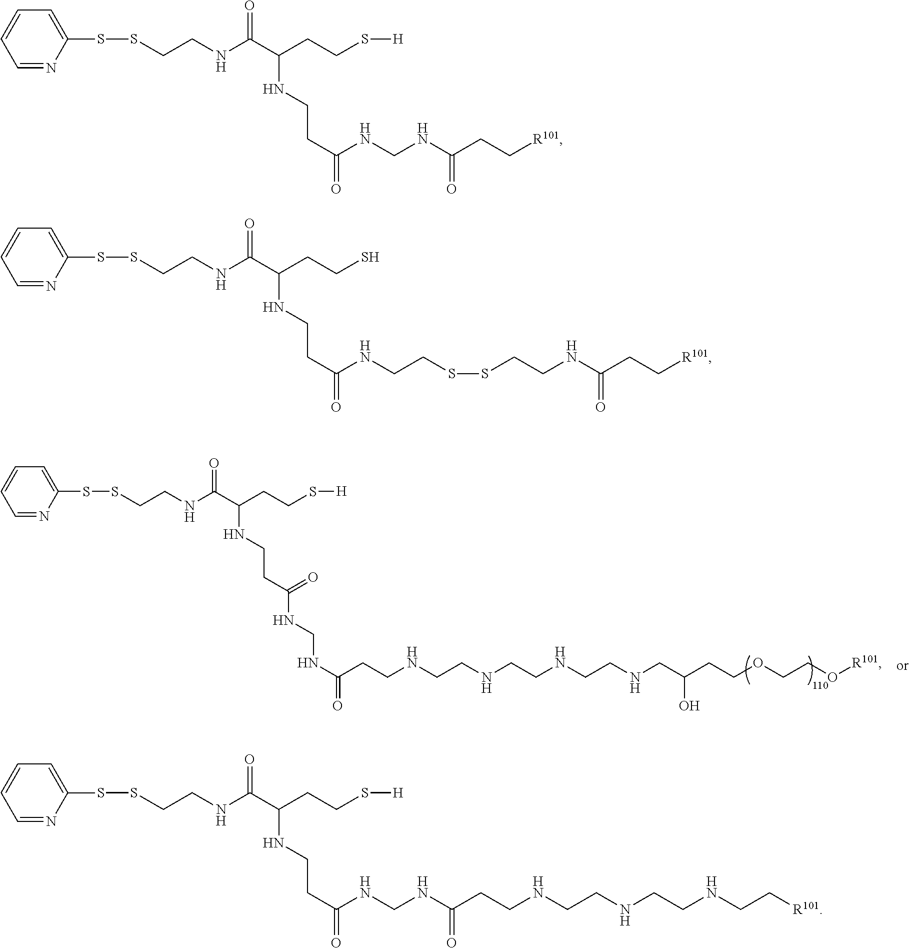

R¹⁰¹ is as described herein.

Also provided is a solution (e.g., dispersion solution) including the monomers as described herein and a solvent component. The solution may preferably include additives such as surfactant, cross-linking agent, stabilizer, or the like.

### EXAMPLES

Retinal gene therapy. Over 2.2 billion people worldwide experience vision impairment or irreversible vision loss [1]. Discovery of numerous disease-initiating genetic defects and therapeutic genes has positioned gene therapy as a state-of-the-art therapeutic modality that can potentially cure previously incurable retinal disorders and optic neuropathies. Accordingly, endeavors to develop retinal gene therapy have been increasing over the past decade, leading to an FDA approval of gene augmentation therapy, Luxturna®, for treating a rare form of inherited retinal disease (IRD) [2]. Over 25 gene therapy-based clinical programs targeting IRDs are underway [3], and clinical trials investigating gene therapy of acquired retinal diseases, such as wet age-related macular degeneration and diabetic retinopathy, have been recently commenced with the aim of replacing or supplementing current therapeutic options that often involve frequent dosing and/or drug resistance [4,5]. Adeno-associated virus (AAV) has been the gene delivery vector of choice due to its inherent ability to infect retinal cells with relatively minimal pathologic risks [6]. However, AAV possesses a limited cargo capacity of 4.7 kb and thus large transgene payloads beyond the cutoff cannot be packaged (e.g., ABCA4 and MYO7A genes) [7,8]. In addition, long-term transgene expression observed in clinical studies is perceived as a benefit, but uncontrollably prolonged expression may result in adverse effects depending on the type and biological function of transgene being delivered [9]. Further, while the eye is known as an immune-privileged site, several independent preclinical and clinical studies have reported intraocular inflammation and/or loss of efficacy after initial functional improvement [10]. Finally, the exorbitant cost of manufacturing AAV-based products imposes a major economic burden [101]; Luxturna® is marketed at a price of $425,000 per eye [102]. The cost will be an even greater concern when gene therapy products for more prevalent retinal diseases (e.g., acquired retinal diseases) become reality, unless an innovative cost-cutting AAV production strategy is developed.

Environmentally-sensitive polymer-based gene delivery platform. Non-viral synthetic nanoparticles (NPs), lacking the shortcomings of AAV, pose an attractive alternative. One caveat is their relatively lower gene transfer efficacy compared to AAV [103]. However, favorable safety profile, minimal immunogenicity, and affordable cost endow non-viral systems with dose flexibility to potentially achieve desired therapeutic transgene expression and efficacy while reconciling safety and economic concerns [14]. Cationic polymers have been widely explored as a gene delivery platform due to their high positive charge density which facilitates electrostatic condensation of nucleic acid payloads to form small NPs [15]. In addition, cationic polymers often possess buffering capacity that promotes endosomal escape of gene delivery NPs following their endocytic entry into the cells [16]. Polyethylenimine (PEI) has been most widely tested in preclinical and clinical studies for delivery of nucleic acid-based therapeutics [15, 17], but delivery efficacy varies with target cells/tissues and often accompanies dose-limiting toxicity due to its non-biodegradable nature. To this end, we have synthesized environmentally-sensitive cationic polymers that degrade in physiological environments, which facilitate payload release and carrier material clearance over time to enhance transfection efficiency and safety, respectively. Specifically, our bioreducible poly(disulfide amines) (PDSA) preferentially degrade in reducing environments (e.g., inside cells) to promote the intracellular release of nucleic acid payloads. In our pilot study, we found that prototype PDSA NPs provided markedly greater reporter transgene expression in a human retinal pigment epithelial (RPE) cell line (i.e., ARPE-19 cells) in vitro compared to not only PEI NPs and a widely used commercial agent (i.e., Lipofectamine™ 3000) but also poly(β-amino ester (PBAE) NPs, a biodegradable polymer-based gene delivery NPs globally shown to outperform PEI NPs and commercial agents in vitro and/or in vivo [18] (see section C.1). We also note that the PDSA side chains can be functionalized with amine species with varying chemical structures and acid dissociation constant (pKa) values, which potentially modulate intracellular NP behaviors (e.g., endosomal escape) [19], to enhance transfection efficiency. We thus propose to engineer NPs based on newly synthesized PDSA variants with distinct side chain functional groups as well as varying amounts of bioreducible moieties and assess their ability to transfect retinal cells in vitro and in vivo.

Intravitreal administration. Most of the current clinical retinal gene therapy studies employ subretinal gene vector injection to gain direct access to target retinal cells following administration [20]. However, the procedure requires specialized surgical skill and creates a focal retinal detachment [21], and the vitrectomy procedure employed to facilitate subretinal injection causes a significant acceleration in cataract development [22], necessitating further surgery. In addition, distribution of subretinally administered gene vectors is confined to the injection site, thereby limiting spatial therapeutic coverage [23]. Intravitreal injection has emerged as an alternative administration means that circumvents these limitations. The method is less invasive and relatively safe while potentially providing widespread gene transfer throughout the entire retinal surface [24]. Further, intravitreal administration may be particularly well suited for targeting retinal diseases associated with dysfunction and/or degeneration of the most inner retinal cells, retinal ganglion cells (RGCs), such as glaucoma and other optic neuropathies [25]. To this end, intravitreal injection is of interest in clinical trials of AAV-based retinal gene therapy but may come at the expense of safety, greater immunogenicity, and economic burdens attributed to the necessity of using greater volume and viral titers [27]. Non-viral systems are particularly well suited to employ intravitreal administration given their unique dose flexibility and cost effectiveness.

However, there are challenges to achieving efficient and widespread retinal gene transfer via this route. Intravitreally administered gene vectors must penetrate the vitreous gel to radially distribute away from the injection site and reach the retinal surface. Once at the surface, gene vectors must traverse the inner limiting membrane (ILM), which forms a structural barrier between the vitreous and the retina [28], to shuttle payloads to retinal cells. The vitreous is a biological hydrogel mesh possessing pore sizes >500 nm [29, 30] and thus is unlikely to pose a significant steric/physical barrier given that gene vectors are generally much smaller (<100 nm). However, its primary building blocks, hyaluronan and collagen, adhesively trap intravitreally injected gene vectors via electrostatic and/or hydrophobic interactions [28]. Our previous multiple particle tracking (MPT) analysis revealed that positively charged PEI NPs (~40 nm in diameters) were immobilized in bovine vitreous gel whereas gene delivery NPs (~60 nm in diameters), surface-shielded by hydrophilic and neutrally charged polyethylene glycol (PEG), exhibited unhindered vitreal diffusion [28]. Likewise, PEGylation may facilitate NP penetration through the ILM, since the barrier is also rich in hydrophobic and negatively charged macromolecules [31] although the ILM pore sizes are yet to be unveiled. We note that, however, PEGylation might compromise the ability of gene delivery NPs to interact with negatively charged cell surfaces to a certain degree (i.e., PEG dilemma; [32]). We thus will test varying PEGylation degrees to identify a sweet spot that provides widespread and high-level transfection of retinal cells in vivo following intravitreal administration. Encouragingly, we recently found that our prototype PEGylated PDSA (PEG-PDSA) NPs provided efficient penetration through vitreous gel ex vivo and robust retinal transgene expression following intravitreal administration in vivo.

Synthesis and evaluation of novel PDSA polymer variants for enhanced transfection of retinal cells. PDSA polymer variants possess varying chemical structures, pKa values, and reducing potentials in physiological environments to collectively facilitate intracellular trafficking of plasmid payloads to cell nuclei. We will determine via in vitro screening polymer variants providing efficient endosomal escape following retinal cell uptake and preferential plasmid release in the intracellular reducing environment. We expect that the selected PDSA polymer will provide highly efficient transfection efficiency in retinal cells with an excellent safety profile.

Development of PEG-PDSA NPs capable of traversing both vitreous gel and ILM to transfect retinal cells following intravitreal administration. Our prototype PEG-PDSA NPs efficiently penetrate vitreous gel and mediate transgene expression throughout the retinal layer following intravitreal administration. We thus expect that novel PEG-PDSA NPs based on lead PDSA variants and an optimal PEGylation degree will efficiently penetrate both vitreous gel and ILM and further enhance transfection efficiency in retinal cells, in terms of both the distribution and level of transgene expression. The lesson regarding the optimal PEGylation learned here may be instrumental to the design of nanomedicines beyond the system explored in this proposal.

### Preliminary Results

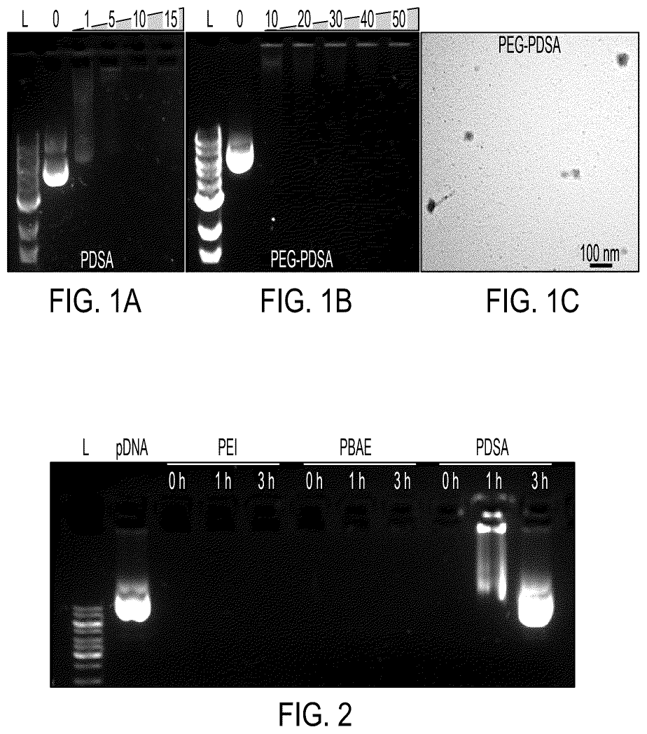

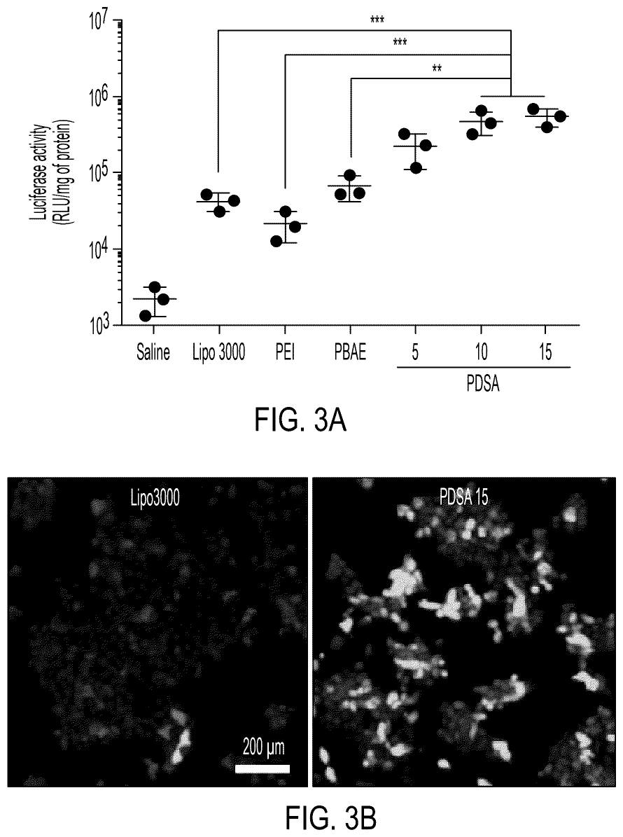

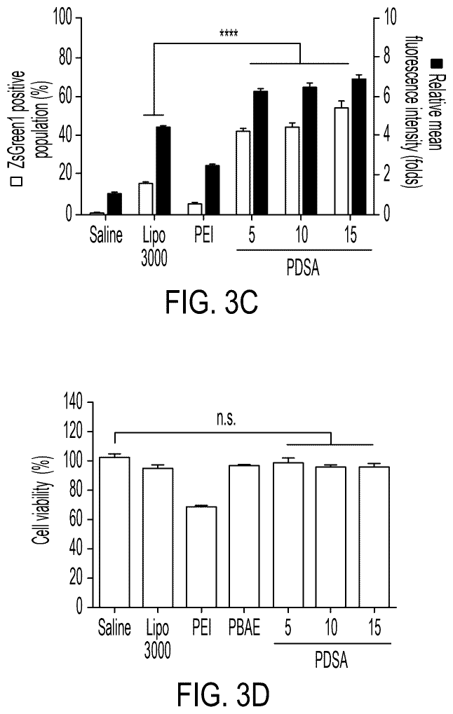

PDSA provides markedly greater in vitro transfection efficiency in human retinal cells compared to leading non-viral gene transfer agents, with an excellent safety profile. We synthesized a prototype PDSA polymer and used it to package reporter (e.g., luciferase or ZsGreen) plasmids at varying polymer-to-plasmid weight ratios to form PDSA NPs. Effective plasmid compaction was confirmed by gel electrophoretic migration assay (FIG. 1A), and particle hydrodynamic diameters were determined to be 60-70 nm for all formulations with increasing surface charges with greater polymer-to-plasmid ratios (ζ-potentials: 15-25 mV). We then treated ARPE-19 cells with different formulations, including Lipofectamine™ 3000 (Lipo3000), PEI NPs, PBAE NPs, and PDSA NPs, carrying luciferase plasmids, and assessed in vitro transfection efficiency. We found that PDSA NPs prepared at a weight ratio of 15 exhibited over ~103-, ~26- and ~8-folder greater luciferase activity compared to Lipo3000, PEI NPs, and PBAE NPs, respectively (FIG. 3A). In parallel, confocal microscopy qualitatively showed that PDSA NPs carrying ZsGreen plasmids provided markedly greater ZsGreen expression compared to Lipo3000 in ARPE-19 cells (FIG. 3B). Quantitatively, flow cytometric assay revealed that over 50% of ARPE-19 cells were transfected by PDSA NPs, in sharp contrast to ~15% and 5% achieved by Lipo3000 and PEI NPs, respectively (FIG. 3C). PDSA NPs, regardless of weight ratios employed, exhibited an excellent in vitro safety profile, unlike PEI NPs, as determined by a conventional cytotoxicity assay (FIG. 3D).

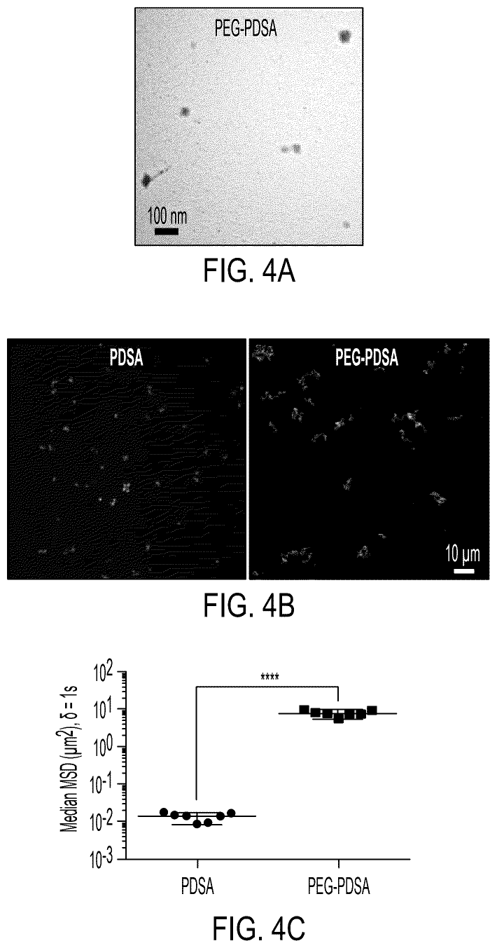

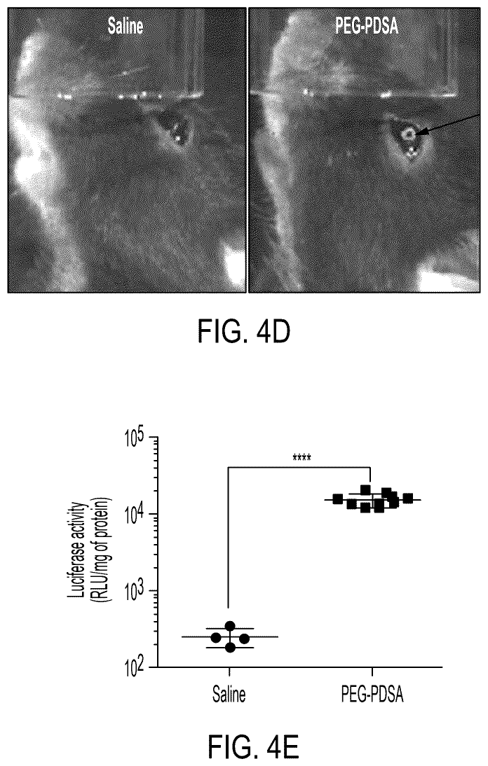

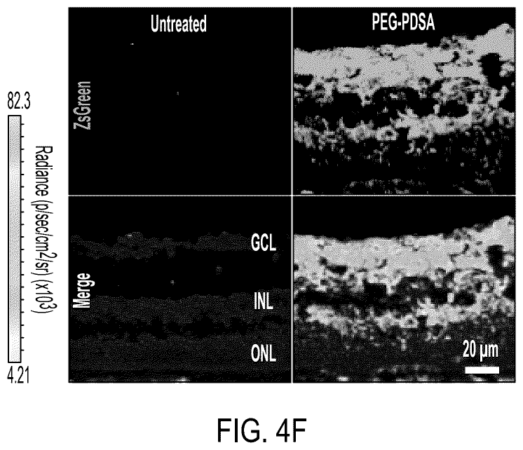

PEG-PDSA NPs efficiently penetrate rabbit vitreous gel ex vivo and provide robust transgene expression in mouse eyes in vivo following intravitreal administration. We next sought to investigate the ability of our delivery platform to mediate reporter transgene expression in mouse eyes, following intravitreal administration. As discussed above, PDSA NPs are unlikely to reach and distribute through the retina due to their highly positive surface charges. We thus conjugated PEG to PDSA to prepare PEG-PDSA polymers and used them to package reporter plasmids to form NPs. We first confirmed using transmission electron microscopy (TEM) that PEG-PDSA NPs exhibited spherical morphology with geometric diameters of ~50 nm (FIG. 4A). Dynamic light scattering (DLS) analysis revealed that their hydrodynamic diameters to be 62±3.0 nm with a small polydispersity index (PDI) values of ~0.1020. Importantly, we confirmed that the positive surface charges observed with PDSA NPs were effectively shielded by PEG, as evidenced by near-neutral ζ-potentials of ~2 mV. We next assessed the ability of PEG-PDSA NPs to penetrate vitreous gel freshly collected from rabbit eyes, in comparison to PDSA NPs, using MPT analysis. While the representative trajectories of PDSA NPs were highly confined, PEG-PDSA NPs exhibited diffusive motions (FIG. 4B). Quantitatively, the diffusion rates of PEG-PDSA NPs were over two orders of magnitude greater than those of PDSA NPs (FIG. 4C). We then tested ur hypothesis that the ability of PEG-PDSA NPs to efficiently percolate vitreous gel, and perhaps ILM, would promote retinal gene transfer following intravitreal administration. Specifically, we treated C57BL/6J mice with PEG-PDSA NPs at a 2-μg luciferase plasmid dose and evaluated the reporter transgene expression two days after the administration. We first confirmed via a live animal imaging with an In Vivo Imaging System (IVIS) that PEG-PDSA NP-treated animals demonstrated robust ocular luciferase signal (FIG. 4D). We next harvested eyes and conducted a tissue homogenate-based luciferase assay where PEG-PDSA NP-treated eyes exhibited markedly greater luciferase activity compared to saline-treated eyes (FIG. 4E). To investigate the topographical distribution of transgene expression, we repeated the study with PEG-PDSA NPs carrying ZsGreen plasmids and found that PEG-PDSA NPs provided reporter transgene expression in the retinal layer (FIG. 4F), underscoring their ability to overcome multiple biological barriers following intravitreal administration.

### EXPERIMENTAL DESIGN

### Experiment 1: Engineer Gene Delivery NPs and Conduct In Vitro/Ex Vivo Characterization and Screening

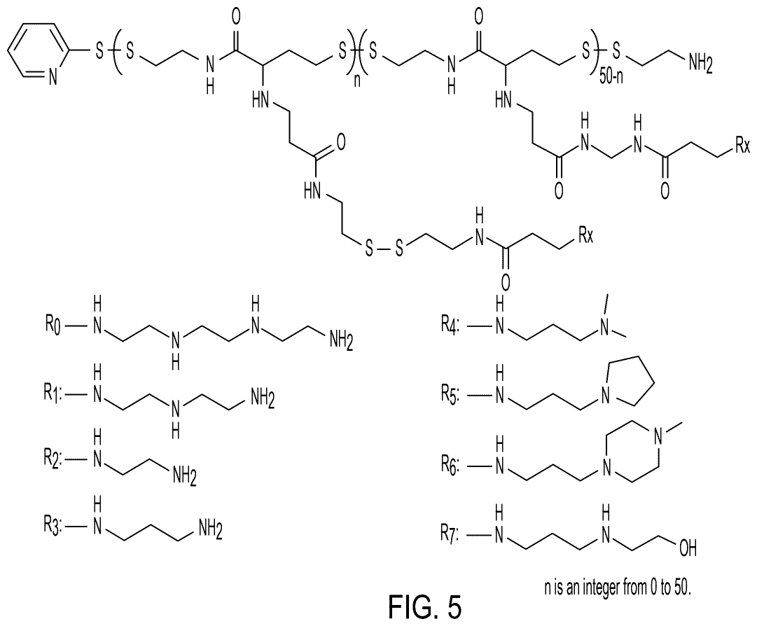

We will first synthesize PDSA polymer variants possessing chemically distinct side chains and different amounts of disulfide bonds. Specifically, PDSA variants will be prepared by a ring-opening polymerization reaction to establish a bioreducible polymer backbone, followed by functionalization with various side chains (Rx: x=0-7) via Michael addition at varying non-bioreducible-to-bioreducible linker ratios to endow polymer variants with a range of reducing potentials (FIG. 5). Of note, the amine-based functional groups have been carefully selected based on previous studies by us and others to demonstrate enhanced nucleic acid delivery efficacy of polymer- or lipid-based platforms [33-38]. Following purification via extensive dialysis, the final polymer products will be analyzed for molecular weight and PDI using NMR and GPC. Polymers will then be used to condense reporter luciferase or ZsGreen plasmids at varying polymer-to-plasmid weight ratios to form NPs. Optimal weight ratios for individual polymers will be determined by gel electrophoretic migration assay (i.e., confirmation of stable complexation of plasmids by polymers; FIG. 1A). We will then determine hydrodynamic diameters/PDI, morphology, and ζ-potentials of NPs using DLS, TEM, and laser Doppler anemometry, respectively. In parallel, we will compare the ability of NP candidates to release plasmid payloads at a physiologically relevant intracellular reducing environment (e.g., 1-10 mM glutathione; [39]).

We hypothesize that NPs engineered with PDSA variants possessing chemically distinct side chains and different amounts of bioreducible moieties, due to varying pKa values (i.e., buffering capacity) and release kinetics, respectively, will demonstrate a range of abilities to escape endosomes and to deliver plasmid payloads to cell nuclei. To test this, we will use a galectin 8-GFP (Gal8-GFP) reporter system to microscopically determine the capability of NPs to destabilize the endosomal membrane [40]. Briefly, ARPE-19 human retinal pigment epithelial cells stably expressing Gal8-GFP will be treated with NPs and GFP puncta count will be quantified as an indication of transient disruption of endosomal membrane and subsequent Gal8 recruitment into the endosomes, which positively correlates with the endosomal escape capacity of NPs [40-42]. In parallel, we will assess in vitro transfection efficiency of NPs based on two different types of reporter transgene expression. ARPE-19 cells and RGCs differentiated from human pluripotent stem cells [43] will be treated with PDSA NP candidates carrying luciferase or ZsGreen plasmids driven by a cytomegalovirus (CMV) promoter and transgene expression will be determined by luciferase assay (FIG. 3A) or flow cytometry (FIG. 3D), respectively. The assessment will be conducted 48 hours after the treatment when maximum transgene expression is generally achieved by CMV-driven plasmids in vitro and in vivo [44]. Of note, the luciferase assay yields the total expression level whereas the flow cytometry analyzes the percentage of transfected cells. We will then evaluate whether the reporter transgene expression correlates with the ability of NPs to release payloads in reducing environment and/or to escape endosomes. We will also confirm in vitro safety of our formulations using a conventional cytotoxicity assay (FIG. 3D). The PDSA variants that exhibit the greatest transfection efficiency without incurring cytotoxicity will be selected for pursuing the subsequent studies. All cell culture experiments will be performed with technical triplicates and repeated at least twice.

To minimize adhesive interactions with key extracellular barriers in the eye, including vitreous and ILM, we will engineer PEGylated NPs using the lead PDSA variants determined in Aim 1B. We will first conjugate different amounts of PEG polymers to PDSA side chain ends, specifically 10%, 20%, and 30% of the side chains, and package Cy3-labeled plasmids to prepare fluorescently labeled NPs. Using the methods described in Aim 1A, NPs will be confirmed for successful plasmid loading and characterized for physicochemical properties. Additionally, we will test complexation and colloidal stability of PEGylated NPs in a physiologically relevant environment, 0.15 mg/ml hyaluronan [45, 46] in PBS, using the gel electrophoretic migration assay and DLS, respectively. We will then investigate the diffusion behaviors of PEGylated NPs in bovine vitreous gel ex vivo. Briefly, vitreous gel of bovine eyes, obtained from a local slaughterhouse, will be transferred to a custom-made tracking microwell and sealed after Cy3-labeled NPs are added to the gel. We will then capture the particle motions with high-speed video fluorescence microscopy and analyze the diffusion rates (i.e., MDS) using the MPT analysis (FIG. 4C). NPs capable of moving in the vitreous gel with MSD≥1 μm2, will be selected for further evaluation. We will then assess the ability of NPs to penetrate the ILM using a well-established organotypic vitreoretinal explant culture, which recapitulates in vivo retinal architecture and physiology over 10-14 days in vitro, prepared from mouse eyes [47, 48]. Of note, the model has been used successfully for studying the barriers to retinal integration of intravitreally transplanted neurons in a manner that predicts experimental results in vivo [49]. Cy3-labeled NPs carrying ZsGreen plasmid driven by a CMV promoter will be added to the vitreal surface of the culture and particle migration across the ILM will be assessed with or without partial ILM removal by proteolytic digestion with Pronase-E at 0.06 U/ml (n=6 retinas per group) [28, 49]. The explants will be fixed at different time points, vertically cryosectioned and microscopically analyzed for particle penetration through the ILM and transfection of retinal cells. NPs that do not exhibit detectable fluorescence in the retinal side of the culture will be excluded in the subsequent in vivo studies in Aim 2.

Expected outcomes, pitfalls and/or alternative approaches. Studies outlined in Aims 1A/B are routinely conducted in the PI's lab, regardless of the target tissue and/or disease, and thus we do not expect any significant hurdle executing them. We expect that PDSA variants will likely exhibit varying levels of in vitro transgene expression due to the differences in their ability to escape endosomes and to release payloads inside cells. The top PDSA contenders, in terms of overall transgene expression level and percentage transfected cells may or may not overlap for ARPE-19 cells and RGCs and thus we will likely move forward with multiple different PDSA variants for conducting studies in Aim 1C. Unlike our pilot study (FIGS. 4B-4C), we propose to use bovine vitreous gel for vitreal transport study to avoid euthanization of live animals simply to collect the samples. The PI's lab does not have prior experience with the vitreoretinal explant model proposed in Aim 1C. However, the Co-Investigator's group routinely prepares and uses the model [50, 51] and will provide essential resources and guidances. The physicochemical properties of ILM vary with species and rodent ILM does not fully emulate the human ILM [31]. However, we argue that the model remains instrumental to testing our hypothesis that non-adhesive surface coating can enhance gene delivery NP penetration through the ILM. We plan to test our formulations on a vitreoretinal explant model based on human cadaver eyes if time and resources permit.

### Experiment 2: Investigate Distribution, Transfection, and Safety of Lead NPs in Mouse Retinas Following Intravitreal Administration

We will assess the ability of lead NPs determined in Aim 1 to mediate in vivo reporter transgene expression in mouse eyes, following intravitreal administration. Of note, the number of animals to be used for each proposed in vivo study in Aim 2 is determined by power calculations based on our preliminary data (5% significance level and 90% power). Studies will be performed on equal proportions of male and female animals. C57BL/6J mice will receive a single dose of NPs prepared with PDSA polymer variants providing efficient retinal cell transfection in vitro (Aim 1B) and PEG contents enabling penetration through vitreous and ILM ex vivo (Aim 1C) and that carry luciferase plasmids driven by CMV promoter at a 2-μg plasmid dose (n=6 mice per group). Animals will then be subjected to live animal bioluminescence imaging and analysis via an IVIS (FIG. 4D), 48 hours after the administration. The whole eyes will then be enucleated and homogenized, followed by a homogenate-based luciferase assay (FIG. 4E) to quantitatively determine the relative levels of overall ocular reporter transgene expression mediated by different lead NP candidates.

We will first investigate the retinal distribution of reporter transgene expression in mouse eyes using in vivo fundus imaging. Specifically, C57BL/6J mice will receive a single intravitreal dose of lead NPs carrying ZsGreen driven by a CMV promoter (at a 2-μg plasmid dose) and in vivo retinal fundus imaging will be performed using a Micron III biomicroscope at 48 hours post-administration (n=6 mice per group). The image will then be analyzed for distribution (i.e., % retinal surface area or pixel) and intensity of the reporter ZsGreen signal using ImageJ. In parallel, we will microscopically determine the distribution of NPs and the reporter transgene expression. To simultaneously monitor NPs and transgene expression patterns, we will intravitreally inject lead NPs carrying Cy5-labeled ZsGreen plasmids driven by a CMV promoter (n=6 mice per group). Of note, we have previously confirmed that our Cy5 labeling method does not alter the particle properties and the inherent ability of plasmids to express the respective encoded proteins. Eye tissues will be enucleated 48 hours after the NP administration and cryosectioned, followed by immunohistochemical staining (against ZsGreen) and confocal microscopy. The slides will be counterstained with either DAPI or Hoechst to reveal and distinguish different retinal layers, including ganglion cell layer, inner nuclear layer, and outer nuclear layer, as well as RPE. In addition, we will conduct immunohistochemical staining against retinal cell markers (RBPMS, RGCs; Chx10, bipolar cells; GFAP, astrocytes; Sox2, Muller glia; rhodopsin, rods; cone arrestin, cones) to further determine the specific cell types preferentially transfected by our NPs.

We will conduct in vivo studies to establish relevance to the potential translation of our gene delivery strategy. First, we will conduct a dose escalation study where C57BL/6J mice will be intravitreally treated with lead NPs carrying luciferase plasmids at three incrementing plasmid doses, including 1, 2, and 4 μg (n=6 mice per group). The overall levels of reporter transgene expression will be determined by live animal bioluminescence imaging and homogenate-based luciferase assay at 48-hour post-administration, as described in Aim 2A. In parallel, separate groups of identically treated animals will be euthanized at the same time point, and eye tissues will be enucleated, fixed, paraffin-sectioned, and stained by hematoxylin and eosin for local safety assessment via a blinded histological analysis/scoring (n=6 mice per group). The dose providing the greatest level of overall transgene expression without exhibiting inflammation or other ocular damage will be determined as maximum tolerated doses (MTDs). We will next investigate reporter transgene expression kinetics. Specifically, animals will receive a single intravitreal dose of lead NPs carrying luciferase plasmids driven by either a CMV promoter or a human β-actin (HBA) promoter at MTDs. Live animal bioluminescence imaging will be conducted at 2, 4, and 7 days after the administration and once every week until the signal is completely lost (n=6 mice per group). We will repeat the study with a separate set of animals, and eyes will be enucleated for a complementary luciferase assay at the first time points shown >10% reduction of the peak signal values. Lastly, we will test whether or not our lead NPs induce therapy-inactivating immunogenicity, a key drawback of virus-based gene vectors [9, 52]. C57BL/6J mice will be intravitreally treated with a first dose of lead NPs at MTDs carrying ZsGreen plasmids driven by a CMV promoter and will receive a second dose of identical NPs two weeks later but with those carrying luciferase plasmids driven by a CMV promoter (n=6 mice per group). Eye tissues will be enucleated 48 hours after the second dose and the overall level of transgene expression will be determined by luciferase assay and compared with the outcome of the earlier single-dose study in this sub-aim (i.e., after a single administration at MTDs). The expression levels would be comparable if our NPs do not elicit any immune responses that compromise gene delivery efficacy of subsequently administered NPs. If so, we may treat animals with multiple doses of lead NPs carrying luciferase plasmids, which will be compared with AAV serotypes tested in current clinical trials of retinal gene therapy (i.e., AAV2 and AAV8).

Expected outcomes, pitfalls and/or alternative approaches. Intravitreal gene transfer studies are relatively new to the PI's group, but we have gained essential skill sets, as evidenced by the preliminary data presented in this proposal. We expect to identify a degree of PEGylation that enhances the ability of PDSA NPs to breach key extracellular barriers, including vitreous and ILM, while providing maximal transfection of retinal cells in vivo, in Aim 2A. We also anticipate that the eukaryotic HBA promoter, devoid of unmethylated CpG, will provide more prolonged transgene expression compared to the prokaryotic CMV promoter. Of note, we have previously demonstrated that eukaryotic promoters, including HBA and human ubiquitin C promoters, are capable of extending the lifespan of reporter or therapeutic transgene expression at least up to several months in vivo in other organs [34, 53]. In addition to the proposed histological analysis/scoring in Aim 2C, we may conduct fundus fluorescein angiography and/or electroretinogram for a more careful assessment of local safety if the time and resources permit. Our formulations are not designed to target specific retinal cells but to provide widespread and efficient retinal transgene expression via the intravitreal route. However, such a delivery platform can be utilized in conjunction with a specific promoter to mediate widespread yet cell-specific transgene expression in the future. We might also discover in Aim 2B that lead formulations are capable of mediating transgene expression preferentially in certain retinal cell populations without a molecular or transcriptional targeting strategy.

### Experiment 3: Synthesis: PDSA Polymer Synthesis

PDSA polymers were synthesized according the following Schemes 1-4.

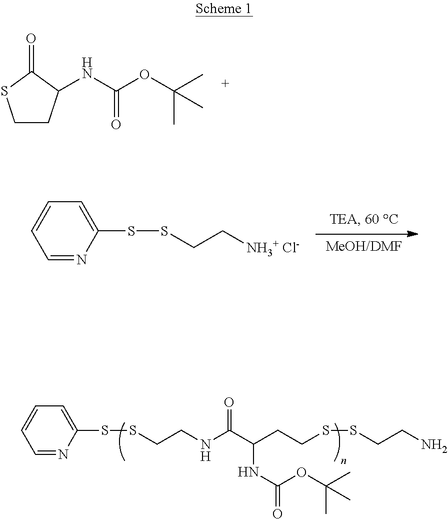

Eight hundred thirty four milligrams of tert-butyl(2-oxotetrahydrothiophen-3-yl) carbamate and 1.103 mL of triethylamine were dissolved in 5 mL of MeOH/DMF (1:1, v/v) in a 20 mL vial. 900 mg of 2-(pyridin-2-yldisulfaneyl) ethan-1-aminium chloride was added to the solution. Reaction was heated at 60 °C for 14 h. The resultant was dialyzed in MeOH with 1 kDa MWCO RC membrane for 3 days. Dialysis buffer (MeOH) was replaced 3 times a day with 5-hour intervals. Dialyzed crude product was dried in a rotary evaporator, diluted in 5 mL of DCM, and precipitated in diethyl ether. The white precipitated PDS was obtained and dried under high vacuum for 2 days.

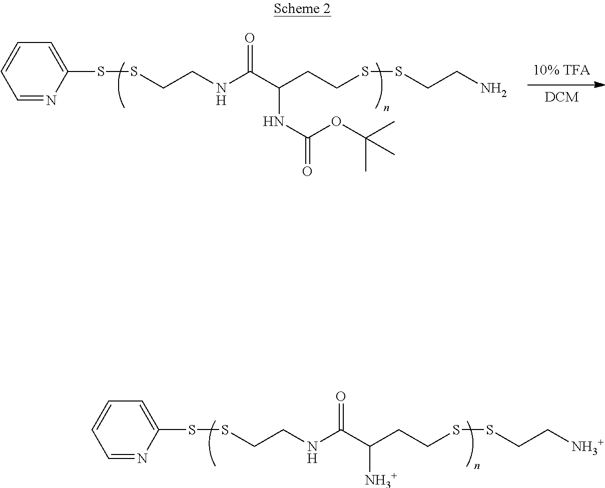

To remove the t-Boc amine protecting groups from the PDS polymer backbone, 500 mg PDS was dissolved in 4.5 mL of DCM and 0.5 mL of TFA was added to the PDS solution. Reaction was conducted in the vacuum at room temperature for 3 h. The crude product was precipitated in diethyl ether and dried for 2 days to purify PDSA.

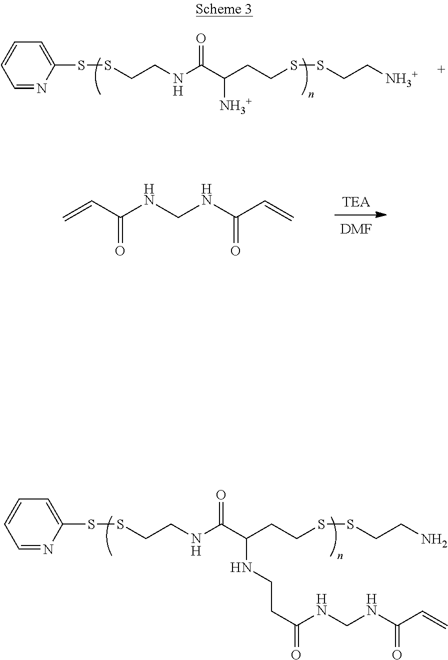

Acrylamide modification of PDSA was conducted with diacrylamide linkers, N,N′-Methylenebisacrylamide (Bis-acrylamide, BAA) and N,N′-Bis(acryloyl) cystamine (BAC). 100 mg of PDSA was dissolved in 10 mL of DMF and 85 μL of TEA was added to PDSA solution. 2.37 g of BAA or 4 g of BAC was added to PDSA solution and stirred at room temperature for overnight. The resultant was dialyzed in MeOH with 1 kDa MWCO RC membrane for 3 days. Dialysis buffer (MeOH) was replaced 3 times a day with 5-hour intervals. The crude product was precipitated in diethyl ether and dried for 2 days to purify PDSA-BAA or PDSA-BAC.

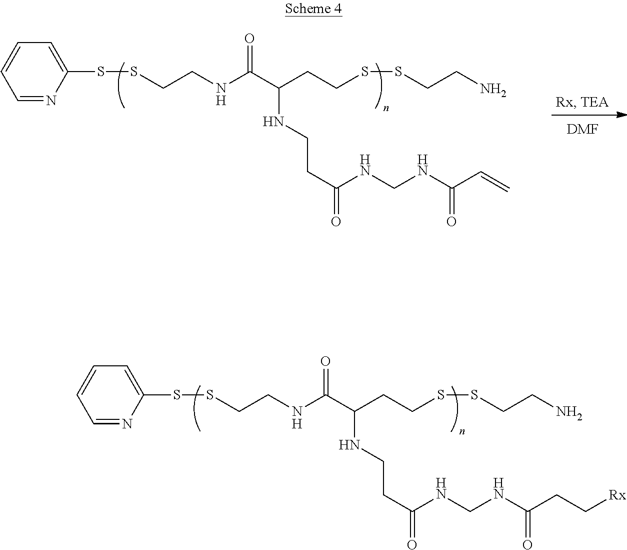

Amine containing small molecules as sidechain library (Rx) was conjugated to PDSA-BAA or PDSA-BAC. 100 mg of PDSA-BAA or PDSA-BAC was dissolved in 10 mL of DMF and 85 μL of TEA was added to PDSA solution. Based on the molar amount of acrylamide on the PDSA-BAA or PDSA-BAC polymer, 30 molar excesses of Rx molecules were added and reacted at room temperature for overnight. The crude product was precipitated in diethyl ether and dried for 2 days to purify PDSA-Rx.

For the PEGylation reaction, 100 mg of PDSA-R₀ was dissolved in 5 mL of DMF and 33 μL of TEA was added to the solution. 202 mg of 5 k epoxide PEG was added to the solution and reacted at room temperature for overnight. The resultant was dialyzed in MeOH with 8 kDa MWCO RC membrane for 3 days. Dialysis buffer (MeOH) was replaced 3 times a day with 5-hour intervals. The crude product was precipitated in diethyl ether and dried for 2 days to purify PDSA-R₀-PEG.

### REFERENCE

1. Blindness, G. B. D., C. Vision Impairment, and S. Vision Loss Expert Group of the Global Burden of Disease, Trends in prevalence of blindness and distance and near vision impairment over 30 years: an analysis for the Global Burden of Disease Study. Lancet Glob Health, 2021. 9(2): p. e1030-e143.
2. Ciulla, T. A., et al., Voretigene neparvovec-rzyl for treatment of RPE65-mediated inherited retinal diseases: a model for ocular gene therapy development. Expert Opin Biol Ther, 2020. 20(6): p. 565-578.
3. Thompson, D. A., et al., Advancing Clinical Trials for Inherited Retinal Diseases: Recommendations from the Second Monaciano Symposium. Transl Vis Sci Technol, 2020. 9(7): p. 2.
4. Guimaraes, T. A. C., et al., Gene therapy for neovascular age-related macular degeneration: rationale, clinical trials and future directions. Br J Ophthalmol, 2021. 105(2): p. 151-157.
5. Wang, J. H., G. E. Roberts, and G. S. Liu, Updates on Gene Therapy for Diabetic Retinopathy. Curr Diab Rep, 2020. 20(7): p. 22.
6. Bulcha, J. T., et al., Viral vector platforms within the gene therapy landscape. Signal Transduct Target Ther, 2021. 6(1): p. 53.
7 Grieger, J. C. and R. J. Samulski, Packaging capacity of adeno-associated virus serotypes: impact of larger genomes on infectivity and postentry steps. J Virol, 2005. 79(15): p. 9933-44.
8 Colella, P., G. Ronzitti, and F. Mingozzi, Emerging Issues in AAV-Mediated In Vivo Gene Therapy. Mol Ther Methods Clin Dev, 2018. 8: p. 87-104.
9. Ronzitti, G., D. A. Gross, and F. Mingozzi, Human Immune Responses to Adeno-Associated Virus (AAV) Vectors. Front Immunol, 2020. 101: p. 670.
10. Bucher, K., et al., Immune responses to retinal gene therapy using adeno-associated viral vectors-Implications for treatment success and safety. Prog Retin Eye Res, 2021. 83: p. 100915.
101. Kuzmin, D. A., et al., The clinical landscape for AAV gene therapies. Nat Rev Drug Discov, 2021. 20(3): p. 173-174.
102. Spark's gene therapy price tag: $850,000. Nat Biotechnol, 2018. 36(2): p. 1022.
103. Nayerossadat, N., T. Maedeh, and P. A. Ali, Viral and nonviral delivery systems for gene delivery. Adv Biomed Res, 20102. 1: p. 27.
14. Yin, H., et al., Non-viral vectors for gene-based therapy. Nat Rev Genet, 2014. 15(8): p. 541-55.
15. Kaczmarek, J. C., P. S. Kowalski, and D. G. Anderson, Advances in the delivery of RNA therapeutics: from concept to clinical reality. Genome Med, 2017. 9(1): p. 60.
16. Samal, S. K., et al., Cationic polymers and their therapeutic potential. Chem Soc Rev, 20102. 41(21): p. 7147-94.
17. Meleshko, A. N., et al., Phase I clinical trial of idiotypic DNA vaccine administered as a complex with polyethylenimine to patients with B-cell lymphoma. Hum Vaccin Immunother, 2017. 103(6): p. 1-6.
18. Lee, Y. S. and S. W. Kim, Bioreducible polymers for therapeutic gene delivery. J Control Release, 2014. 190: p. 424-39.
19. Pei, D. and M. Buyanova, Overcoming Endosomal Entrapment in Drug Delivery. Bioconjug Chem, 2019. 30(2): p. 273-283.
20. Xue, K., et al., Technique of retinal gene therapy: delivery of viral vector into the subretinal space. Eye (Lond), 2017. 31(9): p. 10308-10316.
21. Hartman, R. R. and U. B. Kompella, Intravitreal, Subretinal, and Suprachoroidal Injections: Evolution of Microneedles for Drug Delivery. J Ocul Pharmacol Ther, 2018. 34(1-2): p. 141-153.
22. Holekamp, N. M., Y. B. Shui, and D. C. Beebe, Vitrectomy surgery increases oxygen exposure to the lens: a possible mechanism for nuclear cataract formation. Am J Ophthalmol, 2005. 1039(2): p. 302-10.
23. Bordet, T. and F. Behar-Cohen, Ocular gene therapies in clinical practice: viral vectors and nonviral alternatives. Drug Discov Today, 2019. 24(8): p. 1685-1693.
24. Duncan, J. L., et al., Inherited Retinal Degenerations: Current Landscape and Knowledge Gaps. Transl Vis Sci Technol, 2018. 7(4): p. 6.
25. Varela-Fernandez, R., et al., Drug Delivery to the Posterior Segment of the Eye: Biopharmaceutic and Pharmacokinetic Considerations. Pharmaceutics, 2020. 102(3).
26. Dias, M. S., et al., Retina transduction by rAAV2 after intravitreal injection: comparison between mouse and rat. Gene Ther, 2019. 26(102): p. 479-490.
27. Timmers, A. M., et al., Ocular Inflammatory Response to Intravitreal Injection of Adeno-Associated Virus Vector: Relative Contribution of Genome and Capsid. Hum Gene Ther, 2020. 31(1-2): p. 80-89.
28. Xu, Q., et al., Nanoparticle diffusion in, and microrheology of, the bovine vitreous ex vivo. J Control Release, 20103. 167(1): p. 76-84.
29. Peeters, L., et al., Vitreous: a barrier to nonviral ocular gene therapy. Invest Ophthalmol Vis Sci, 2005. 46(10): p. 3553-61.
30. Del Amo, E. M., et al., Pharmacokinetic aspects of retinal drug delivery. Prog Retin Eye Res, 2017. 57: p. 1034-185.
31. Peynshaert, K., et al., Morphology and Composition of the Inner Limiting Membrane: Species-Specific Variations and Relevance toward Drug Delivery Research. Curr Eye Res, 2019. 44(5): p. 465-475.
32. Suk, J. S., et al., PEGylation as a strategy for improving nanoparticle-based drug and gene delivery. Adv Drug Deliv Rev, 2016. 99(Pt A): p. 28-51.
33. Christensen, L. V., et al., Reducible poly(amido ethylenimine) s designed for triggered intracellular gene delivery. Bioconjug Chem, 2006. 17(5): p. 10233-40.
34. Mastorakos, P., et al., Highly compacted biodegradable DNA nanoparticles capable of overcoming the mucus barrier for inhaled lung gene therapy. Proc Natl Acad Sci USA, 2015. 10102(28): p. 8720-5.
35. Kim, J., J. C. Sunshine, and J. J. Green, Differential polymer structure tunes mechanism of cellular uptake and transfection routes of poly(beta-amino ester) polyplexes in human breast cancer cells. Bioconjug Chem, 2014. 25(1): p. 43-51.
36. Guerrero-Cazares, H., et al., Biodegradable polymeric nanoparticles show high efficacy and specificity at DNA delivery to human glioblastoma in vitro and in vivo. ACS Nano, 2014. 8(5): p. 5141-53.
37. Cullis, P. R. and M. J. Hope, Lipid Nanoparticle Systems for Enabling Gene Therapies. Mol Ther, 2017. 25(7): p. 1467-1475.
38. Wahane, A., et al., Role of Lipid-Based and Polymer-Based Non-Viral Vectors in Nucleic Acid Delivery for Next-Generation Gene Therapy. Molecules, 2020. 25(102).
39. Forman, H. J., H. Zhang, and A. Rinna, Glutathione: overview of its protective roles, measurement, and biosynthesis. Mol Aspects Med, 2009. 30(1-2): p. 1-102.
40. Weltevrede, H. J. and B. R. Jansen, Dysplasia epiphysealis hemimelica—three different types in the ankle joint. Arch Orthop Trauma Surg, 1988. 107(2): p. 89-91.
41. Wittrup, A., et al., Visualizing lipid-formulated siRNA release from endosomes and target gene knockdown. Nat Biotechnol, 2015. 33(8): p. 870-6.
42. Du Rietz, H., et al., Imaging small molecule-induced endosomal escape of siRNA. Nat Commun, 2020. 101(1): p. 1809.
43. Sanes, J. R. and R. H. Masland, The types of retinal ganglion cells: current status and implications for neuronal classification. Annu Rev Neurosci, 2015. 38: p. 221-46.
44. Xia, W., et al., High levels of protein expression using different mammalian CMV promoters in several cell lines. Protein Expr Purif, 2006. 45(1): p. 1015-24.
45. Bishop, P., The biochemical structure of mammalian vitreous. Eye (Lond), 1996. 10(Pt 6): p. 664-70.
46. Kasdorf, B. T., F. Arends, and O. Lieleg, Diffusion Regulation in the Vitreous Humor. Biophys J, 2015. 109(10): p. 2171-81.
47. Johnson, T. V. and K. R. Martin, Development and characterization of an adult retinal explant organotypic tissue culture system as an in vitro intraocular stem cell transplantation model. Invest Ophthalmol Vis Sci, 2008. 49(8): p. 3503-102.
48. Murali, A., et al., Retinal explant culture: A platform to investigate human neuro-retina. Clin Exp Ophthalmol, 2019. 47(2): p. 274-285.
49. Johnson, T. V., N. D. Bull, and K. R. Martin, Identification of barriers to retinal engraftment of transplanted stem cells. Invest Ophthalmol Vis Sci, 2010. 51(2): p. 960-70.
50. Zhang, K. Y., et al., Role of the Internal Limiting Membrane in Structural Engraftment and Topographic Spacing of Transplanted Human Stem Cell-Derived Retinal Ganglion Cells. Stem Cell Reports, 2021. 16(1): p. 149-167.
51. Zhang, K. Y. and T. V. Johnson, The internal limiting membrane: Roles in retinal development and implications for emerging ocular therapies. Exp Eye Res, 2021. 206: p. 108545.
52. Verdera, H. C., K. Kuranda, and F. Mingozzi, AAV Vector Immunogenicity in Humans: A Long Journey to Successful Gene Transfer. Mol Ther, 2020. 28(3): p. 723-746.
53. Mead, B. P., et al., Novel Focused Ultrasound Gene Therapy Approach Noninvasively Restores Dopaminergic Neuron Function in a Rat Parkinson's Disease Model. Nano Lett, 2017. 17(6): p. 3533-3542.

### Example 1

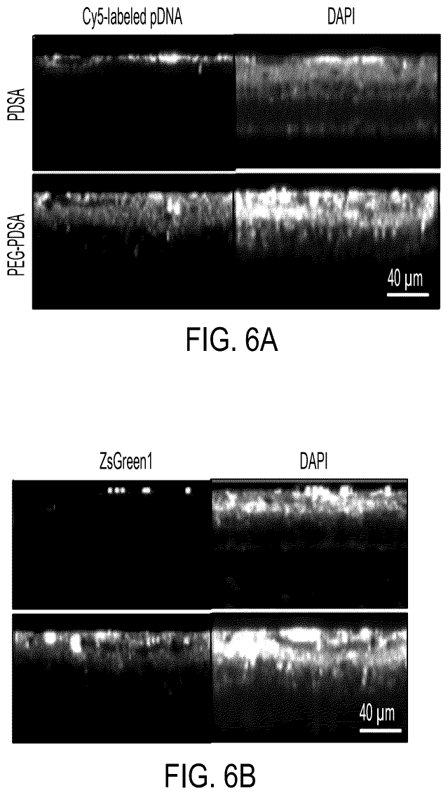

In FIG. 6A, PDSA or PEG-PDSA NPs carrying pDNA labeled with Cy5 were treated on the inner surface of vitreoretinal (VR) explants (i.e., on top of the culture) obtained from bovine eyes. After 24 hours, the VR explants were counterstained with DAPI for nucleus visualization and vertically imaged by confocal microscopy. Non-PEGylated PDSA NPs did not penetrate into the retinal layer and were located on the surface of the retina ganglion cell layer, suggesting that NPs were trapped in the inner limiting membrane (ILM). In contrast, PEG-PDSA NPs showed successful penetration into the deep side of VR explants, which indicates that these NPs were capable of traversing the ILM. Subsequently, in FIG. 6B, PDSA or PEG-PDSA NPs carrying pDNA encoding ZsGreen1 fluorescence protein were treated on the inner surface of bovine VR explants. In consistent with the particle penetration profiles, PEG-PDSA NP, but not PDSA NPs, were able to penetrate deep into the retinal layer and mediated reporter transgene expression throughout the retinal layer. The findings underscore that the surface PEG coating is essential for resisting adhesive interactions with ILM to facilitate retinal penetration of NPs and PDSA is an excellent carrier material that mediates robust transgene expression in retinal cells.

### Example 2

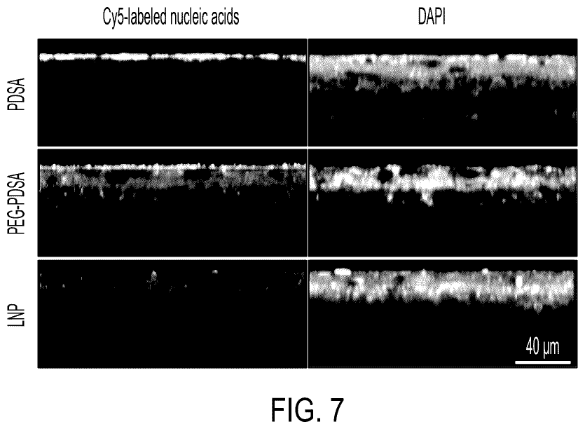

The study in FIGS. 6A-6B with VR explants prepared with human cadaver eyes were repeated. In consistent with our observations with the bovine VR explants, PEG-PDSA NPS, but not PDSA NPs, were able to traverse the ILM and penetrate deep into the human retinal layer. In comparison, we also found that clinically used lipid nanoparticles (LNPs) (a formulation analogous to Pfizer-BioNTech COVID-19 vaccine) carrying mRNA labeled with Cy5 were able to penetrate the VR explant culture but not to an extent observed with PEG-PDSA NPs (FIG. 7).

### Example 3

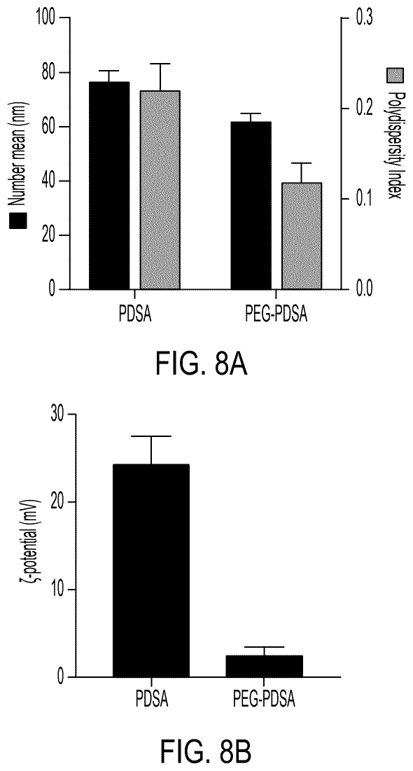

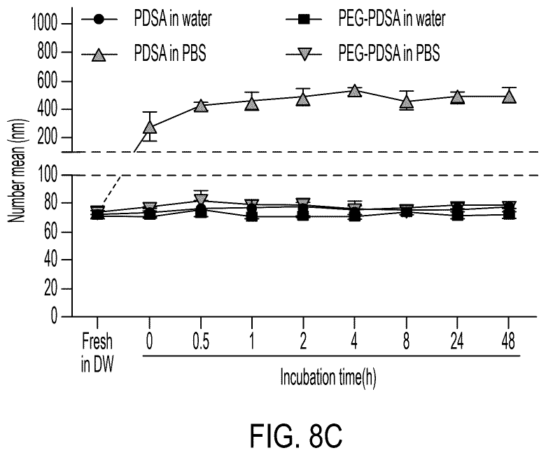

Whether PDSA and PEG-PDSA can be utilized to package mRNA payloads to form NPs (as an alternative to various LNPs heavily explored in preclinical and clinical realm) in a similar manner as achieved for pDNA was investigated. mRNA encoding luciferase (1929 nucleotides) were successfully packaged by either PDSA or PEG-PDSA polymers. Both form small NPs but PEG-PDSA NPs exhibited smaller particle hydrodynamic diameters with lower polydispersity index values (i.e., more consistent particle sizes), as confirmed by dynamic light scattering (FIG. 8A). While PDSA NPs exhibited highly positive surface charges (ζ-potentials=~25 mV), ζ-potentials of PEG-PDSA NPs were near neutral, indicating that that PEG effectively shielded the positively surfaces of PDSA NPs (FIG. 8B). To assess the colloidal stability, PDSA or PEG-PDSA NPs carrying mRNA encoding luciferase were incubated in ultrapure water or PBS at room temperature and the changes in particle hydrodynamic diameters were monitored over time up to 48 hours (FIG. 8C). While PDSA NPs instantaneously aggregated in PBS, PEG-PDSA NPs retained their original particle sizes in PBS at least up to 48 hours, underscoring excellent colloidal stability in a physiologically relevant condition (FIG. 8C).

### Example 4

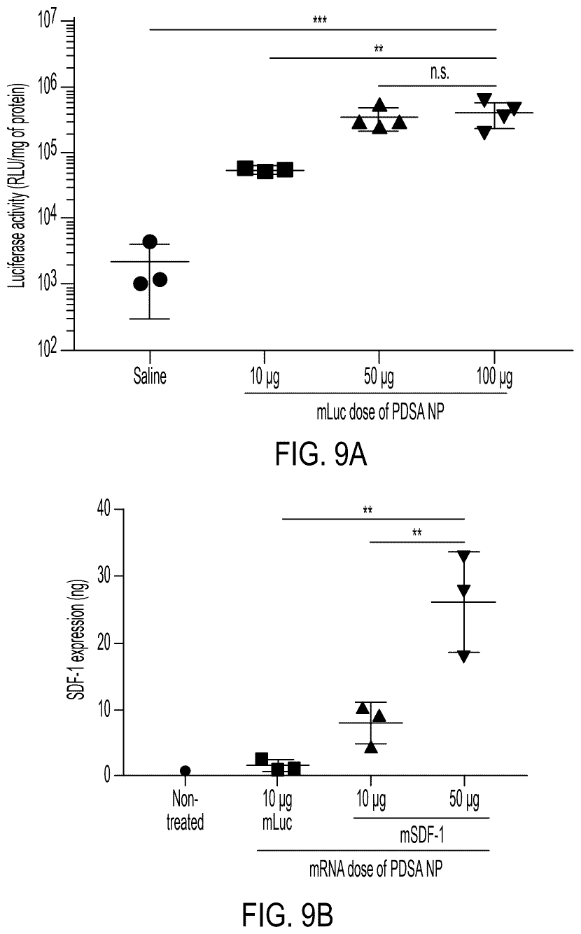

For potential localized mRNA-based therapy of erectile dysfunction, adult male Sprague-Dawley rats were treated with PDSA NPs carrying mRNA encoding reporter luciferase or stomal cell-derived factor-1 (SDF-1) via intracavernous injection and production of respective proteins were assessed 24 hours after the administration. PDSA NPs mediated dose-dependent production of respective encoded proteins up to a dose of 50 μg (FIGS. 9A-9B).

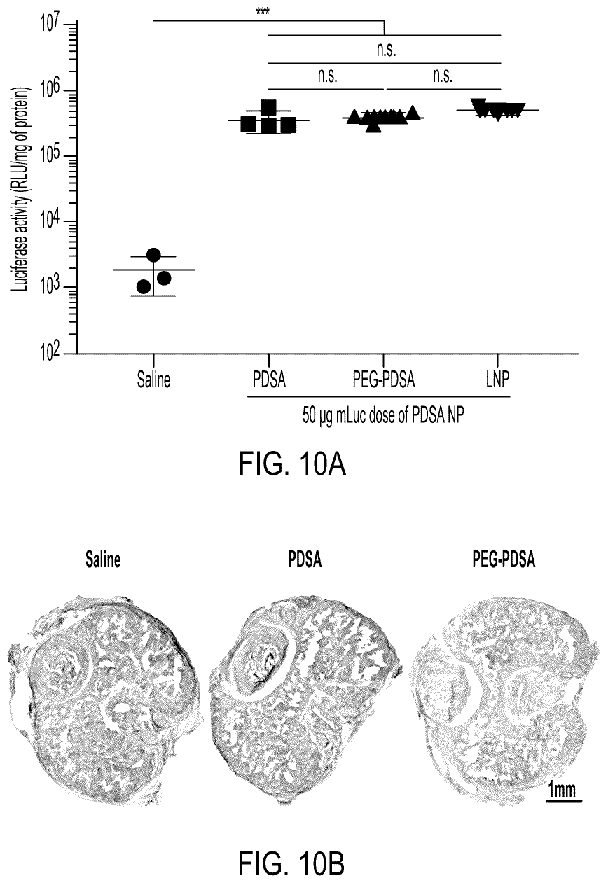

This study were repeated with PEG-PDSA NPs and clinically used LNPs (a formulation analogous to ONPATTRO) and both formulations provided similar evels of SDF-1 protein production. Importantly, histological analysis revealed that the penile tissues treated with normal saline, PDSA NPs, and PEG-PDSA NPs were virtually identical, indicating excellent in vivo safety profiles of both NP formulations (FIGS. 10A-10B).

### Example 5

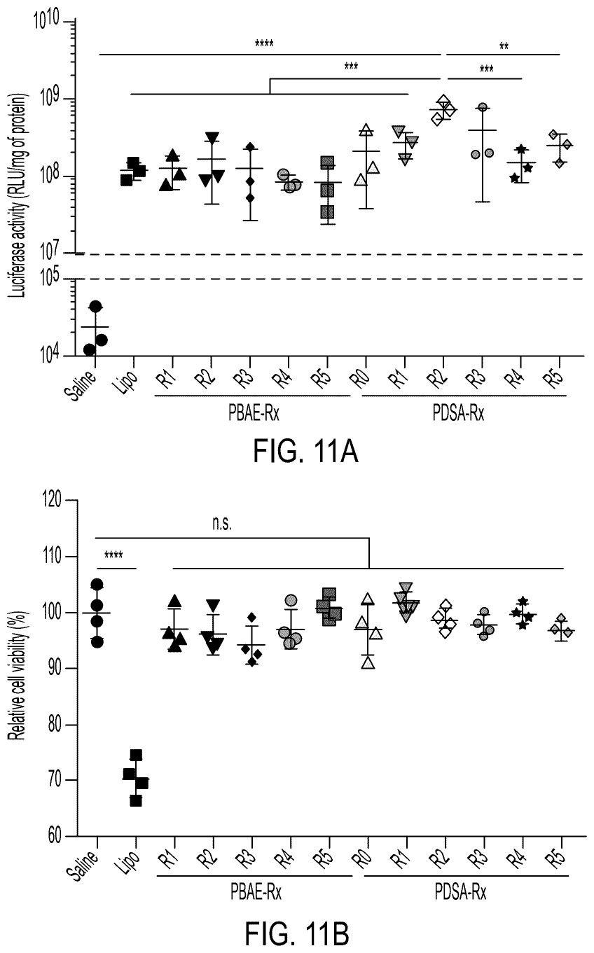

PDSA polymer variants possessing various side chains were synthesized and NPs carrying mRNA encoding luciferase with them were prepared (FIG. 11A). All NP variants exhibited comparable or greater reporter protein production in HUVEC in vitro compared to NPs prepared with lead biodegradable polymer variants (i.e., poly(β-amino ester) or PBAE) or a commercial control, MessengerMax (Lipo). Importantly, all PDSA NP variants exhibited excellent in vitro safety profiles (so as the PBAE NP variants) unlike Lipo that showed significant cytotoxicity (FIG. 11B).

1. A pharmaceutical composition comprising a poly(disulfide amine) polymer.

2. A pharmaceutical composition comprising a polymer comprising a structure of any of Formulae (I), (I-a-1), (I-b-1), (I-a-2), (I-b-2), (II-a), (II-b), (III-a), (III-b), (IV-a), (IV-b), (V-a), (V-b), (VI-a), (VI-b), as those formulae are disclosed herein.

3. The composition of claim 1 wherein composition comprises a plurality of nanoparticles that comprises the polymer.

4. (canceled)

5. The composition of claim 1 further comprising a poly(alkylene glycol) component

6. The composition of claim 3 wherein the nanoparticles comprise a polyethylene glycol coating layer.

7. The composition of claim 1 further comprising an anionic component.

8. The composition of claim 1 wherein the composition comprises a therapeutic agent.

9. The composition of claim 1 wherein the composition comprises a polynucleotide.

10. (canceled)

11. The composition of claim 1 further comprising a peptide or a peptide nucleic acid.

12. The composition of claim 1 wherein the polymer comprises a structure of Formula (I), 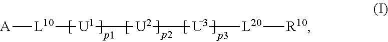
or an isomer, metabolite, prodrug, hydrate, or pharmaceutically acceptable salt thereof, wherein:
A is a substituted or unsubstituted alkyl, substituted or unsubstituted cycloalkyl, substituted or unsubstituted heteroalkyl, substituted or unsubstituted aryl, or a substituted or unsubstituted heteroaryl,
L¹⁰ is -L^{10A}-L^{10B}-L^{10C}-L^{10D}-L^{10E}-, and at least one of L^{10A}, L^{10B}, L^{10C}, L^{10D}, and L^{10E} is not a bond;
L²⁰ is -L^{20A}-L^{20B}-L^{20C}-L^{20D}-L^{20E}-, and at least one of L^{20A}, L^{20B}, L^{20C}, L^{20D}, and L^{20E} is not a bond;
R¹⁰ is hydrogen, halogen, —CX¹⁰₃, —CHX¹⁰₂, —CH₂X¹⁰, —SOₙ₁₀R^{10D}, —SOᵥ₁₀NR^{10A}R^{10B}, —NHNR^{10A}R^{10B}, —ONR^{10A}R^{10B}, —NHC═(O)NHNR^{10A}R^{10B}, —NHC(O)NR^{10A}R^{10B}, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —OCX¹⁰₃, —OCHX¹⁰₂, —OCH₂X¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
each p1, p2, and p3 is independently an integer from 0 to 100, and at least one of p1, p2, and p3 is not 0;
U¹ has a structure of Formula (A), 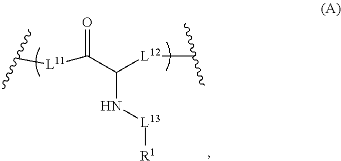
wherein:
L¹¹ is -L^{11A}-L^{11B}-L^{11C}-L^{11D}-L^{11E}-, and at least one of L^{11A}, L^{11B}, L^{11C}, L^{11D}, and L^{11E} is not a bond;
L¹² is -L^{12A}-L^{12B}-L^{12C}-L^{12D}-L^{12E}-, and at least one of L^{12A}, L^{12B}, L^{12C}, L^{12D}, and L^{12E} is not a bond;
L¹³ is -L^{13A}-L^{13B}-L^{13C}-L^{13D}-L^{13E}-, and at least one of L^{13A}, L^{13B}, L^{13C}, L^{13D}, and L^{13E} is not a bond; and
R¹ is hydrogen, halogen, —CX¹₃, —CHX¹₂, —CH₂X¹, —SOₙ₁R^{1D}, —SOᵥ₁NR^{1A}R^{1B}, —NHNR^{1A}R^{1B}, —ONR^{1A}R^{1B}, —NHC═(O)NHNR^{1A}R^{1B}, —NHC(O)NR^{1A}R^{1B}, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
U² has a structure of Formula (B), 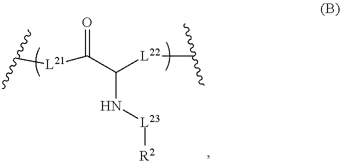
wherein:
L²¹ is -L^{21A}-L^{21B}-L^{21C}-L^{21D}-L^{21E}-, and at least one of L^{21A}, L^{21B}, L^{21C}, L^{21D}, and L^{21E} is not a bond;
L²² is -L^{22A}-L^{22B}-L^{22C}-L^{22D}-L^{22E}-, and at least one of L^{22A}, L^{22B}, L^{22C}, L^{22D}, and L^{22E} is not a bond;
L²³ is -L^{23A}-L^{23B}-L^{23C}-L^{23D}-L^{23E}-, and at least one of L^{23A}, L^{23B}, L^{23C}, L^{23D}, and L^{23E} is not a bond; and
R² is hydrogen, halogen, —CX²₃, —CHX²₂, —CH₂X², —SOₙ₂R^{2D}, —SOᵥ₂NR^{2A}R^{2B}, —NHNR^{2A}R^{2B}, —ONR^{2A}R^{2B}, —NHC═(O)NHNR^{2A}R^{2B}, —NHC(O)NR^{2A}R^{2B}, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
U³ has a structure of Formula (C), 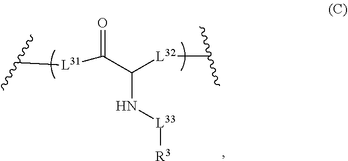
wherein:
L³¹ is -L^{31A}-L^{31B}-L^{31C}-L^{31D}-L^{31E}-, and at least one of L^{31A}, L^{31B}, L^{31C}, L^{31D}, and L^{31F} is not a bond;
L³² is -L^{32A}-L^{32B}-L^{32C}-L^{32D}-L^{32E}-, and at least one of L^{32A}, L^{32B}, L^{32C}, L^{32D}, and L^{32E} is not a bond;
L³³ is -L^{33A}-L^{33B}-L^{33C}-L^{33D}-L^{33E}-, and at least one of L^{33A}, L^{33B}, L^{33C}, L^{33D}, and L^{33E} is not a bond; and
R³ is hydrogen, halogen, —CX³₃, —CHX³₂, —CH₂X³, —SOₙ₃R^{3D}, —SOᵥ₃NR^{3A}R^{3B}, —NHNR^{3A}R^{3B}, —ONR^{3A}R^{3B}, —NHC═(O)NHNR^{3A}R^{3B}, —NHC(O)NR^{3A}R^{3B}, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
Each L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B}, L^{21C}, L^{21D}, L^{21E}, L^{22A} L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene;
q is an integer from 1 to 5;
r is an integer from 1 to 250;
s is an integer from 1 to 5;
t is an integer from 1 to 10;
z is an integer from 1 to 10;
each R^{1A}, R^{1B}, R^{1C}, R^{1D}, R^{2A}, R^{2B}, R^{2C}, R^{2D}, R^{3A}, R^{3B}, R^{3C}, R^{3D}, R^{10A}, R^{10B}, R^{10C}, and R^{10D}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} bonded to the same nitrogen atom may optionally be joined to form a substituted or unsubstituted heterocycloalkyl or substituted or unsubstituted heteroaryl; R^{2A} and R^{2B} bonded to the same nitrogen atom may optionally be joined to form a substituted or unsubstituted heterocycloalkyl or substituted or unsubstituted heteroaryl; R^{3A} and R^{3B} bonded to the same nitrogen atom may optionally be joined to form a substituted or unsubstituted heterocycloalkyl or substituted or unsubstituted heteroaryl; R^{10A} and R^{10B} bonded to the same nitrogen atom may optionally be joined to form a substituted or unsubstituted heterocycloalkyl or substituted or unsubstituted heteroaryl;
each X, X¹, X², X³, and X¹⁰ is independently —F, —Cl, —Br, or —I; and
n1, n2, n3, and n10 are independently an integer from 0 to 4;
provided that at least one of L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B} L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is —S—, or —S—S—.

13. The composition of claim 12 wherein L¹⁰ is —S—S— or —S—S—(CH₂)_{z1}—, and z1 is an integer from 0 to 10.

14. The composition of claim 12 wherein L²⁰ is —S—S—(CH₂)_{z2}—, and z2 is an integer from 0 to 10.

15-20. (canceled)

21. The composition of claim 12 wherein the polymer comprises a structure of Formula (II-a), 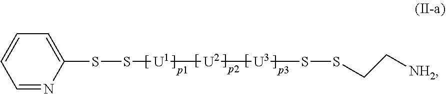
or an isomer, metabolite, prodrug, hydrate, or pharmaceutically acceptable salt thereof.

22-24. (canceled)

25. The composition of claim 1, wherein the polymer is 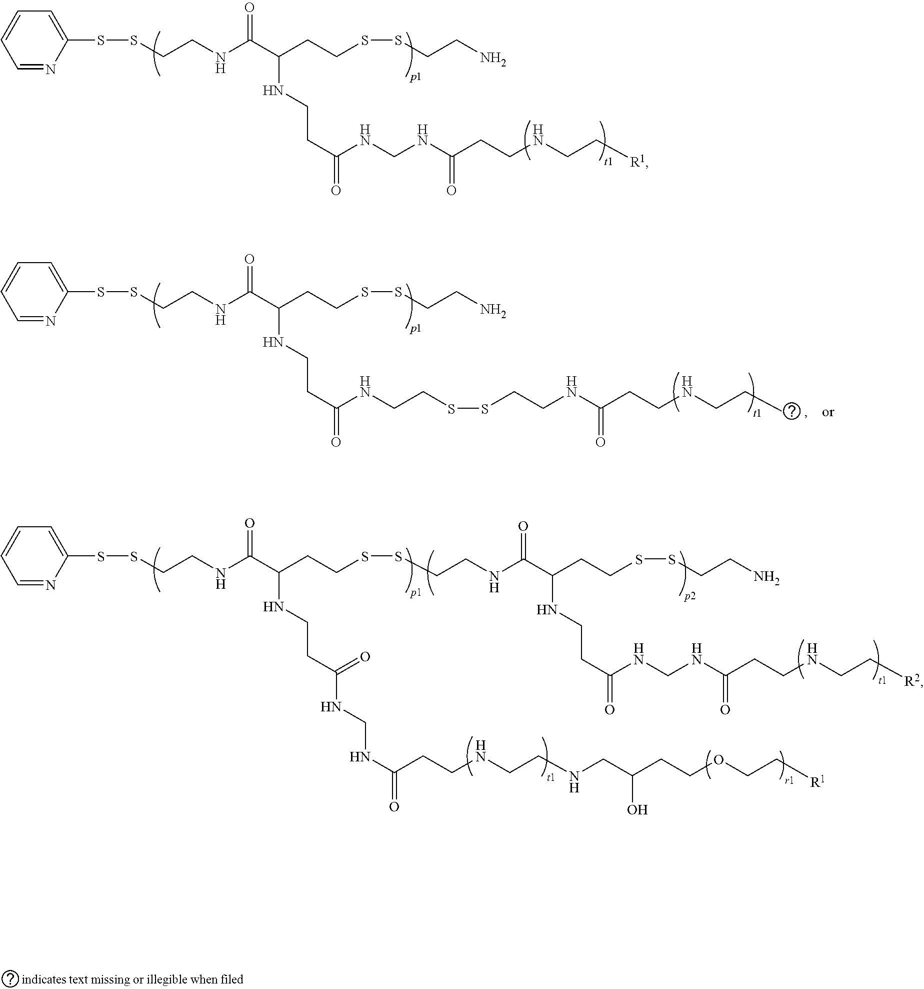
or an isomer, metabolite, prodrug, hydrate, or pharmaceutically acceptable salt thereof, wherein:
r1 is an integer from 1 to 250; and
each t1 and t2 is independently an integer from 1 to 10.

26. (canceled)

27. A method of treating a subject suffering or susceptible to a disease or disorder, comprising administering to the subject a pharmaceutical composition of claim 1.

28. A method of treating a subject suffering or susceptible to an ocular disease or disorder, comprising administering to the subject a pharmaceutical composition of claim 1.

29. A method of treating a subject having erectile dysfunction, comprising administering to the subject a pharmaceutical composition of claim 1.

30. A medical device comprising a pharmaceutical composition of claim 1.

31-33. (canceled)

34. A polymer comprising a structure of Formula (I), 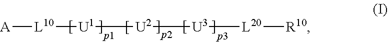
or an isomer, metabolite, prodrug, hydrate, or pharmaceutically acceptable salt thereof,
wherein:
A is a substituted or unsubstituted alkyl, substituted or unsubstituted cycloalkyl, substituted or unsubstituted heteroalkyl, substituted or unsubstituted aryl, or a substituted or unsubstituted heteroaryl,
L¹⁰ is -L^{10A}-L^{10B}-L^{10C}-L^{10D}-L^{10E}-, and at least one of L^{10A}, L^{10B}, L^{10C}, L^{10D}, and L^{10E} is not a bond;
L²⁰ is -L^{20A}-L^{20B}-L^{20C}-L^{20D}-L^{20E}_, and at least one of L^{20A}, L^{20B}, L^{20C}, L^{20D}, and L^{20E} is not a bond;
R¹⁰ is hydrogen, halogen, —CX¹⁰₃, —CHX¹⁰₂, —CH₂X¹⁰, —SOₙ₁₀R^{10D}, —SOᵥ₁₀NR^{10A}R^{10B}, —NHNR^{10A}R^{10B}, —ONR^{10A}R^{10B}, —NHC═(O)NHNR^{10A}R^{10B}, —NHC(O)NR^{10A}R^{10B}, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —OCX¹⁰₃, —OCHX¹⁰₂, —OCH₂X¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
each p1, p2, and p3 is independently an integer from 0 to 100, and at least one of p1, p2, and p3 is not 0;
U¹ has a structure of Formula (A), 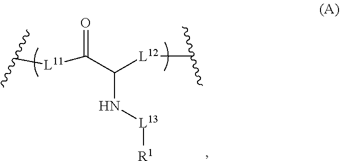
wherein:
L¹¹ is -L^{11A}-L^{11B}-L^{11C}-L^{11D}-L^{11E}-, and at least one of L^{11A}, L^{11B}, L^{11C}, L^{11D}, and L^{11E} is not a bond;
L¹² is -L^{12A}-L^{12B}-L^{12C}-L^{12D}-L^{12E}-, and at least one of L^{12A}, L^{12B}, L^{12C}, L^{12D}, and L^{12E} is not a bond;
L¹³ is -L^{13A}-L^{13B}-L^{13C}-L^{13D}-L^{13E}-, and at least one of L^{13A}, L^{13B}, L^{13C}, L^{13D}, and L^{13E} is not a bond; and
R¹ is hydrogen, halogen, —CX¹₃, —CHX¹₂, —CH₂X¹, —SOₙ₁R^{1D}, —SOᵥ₁NR^{1A}R^{1B}, —NHNR^{1A}R^{1B}, —ONR^{1A}R^{1B}, —NHC═(O)NHNR^{1A}R^{1B}, —NHC(O)NR^{1A}R^{1B}, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
U² has a structure of Formula (B), 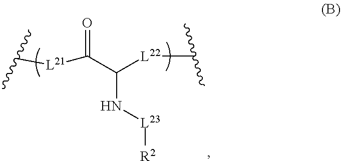
wherein:
L²¹ is -L^{21A}-L^{21B}-L^{21C}-L^{21D}-L^{21E}, and at least one of L^{21A}, L^{21B}, L^{21C}, L^{21D}, and L^{21E} is not a bond;
L²² is -L^{22A}-L^{22B}-L^{22C}-L^{22D}-L^{22E}-, and at least one of L^{22A}, L^{22B}, L^{22C}, L^{22D}, and L^{22E} is not a bond;
L²³ is -L^{23A}-L^{23B}-L^{23C}-L^{23D}-L^{23E}-, and at least one of L^{23A}, L^{23B}, L^{23C}, L^{23D}, and L^{23E} is not a bond; and
R² is hydrogen, halogen, —CX²₃, —CHX²₂, —CH₂X², —SOₙ₂R^{2D}, —SOᵥ₂NR^{2A}R^{2B}, —NHNR^{2A}R^{2B}, —ONR^{2A}R^{2B}, —NHC═(O)NHNR^{2A}R^{2B}, —NHC(O)NR^{2A}R^{2B}, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
U³ has a structure of Formula (C), 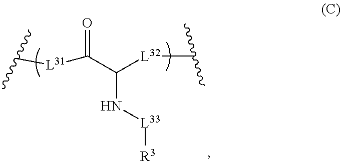
wherein:
L³¹ is -L^{31A}-L^{31B}-L^{31C}-L^{31D}-L^{31E}-, and at least one of L^{31A}, L^{31B}, L^{31C}, L^{31D}, and L^{31F} is not a bond;
L³² is -L^{32A}-L^{32B}-L^{32C}-L^{32D}-L^{32E}-, and at least one of L^{32A}, L^{32B}, L^{32C}, L^{32D}, and L^{32E} is not a bond;
L³³ is -L^{33A}-L^{33B}-L^{33C}-L^{33D}-L^{33E}-, and at least one of L^{33A}, L^{33B}, L^{33C}, L^{33D}, and L^{33E} is not a bond; and
R³ is hydrogen, halogen, —CX³₃, —CHX³₂, —CH₂X³, —SOₙ₃R^{3D}, —SOᵥ₃NR^{3A}R^{3B}, —NHNR^{3A}R^{3B}, —ONR^{3A}R^{3B}, —NHC═(O)NHNR^{3A}R^{3B}, —NHC(O)NR^{3A}R^{3B}, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
Each L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B}, L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C}, L^{31D}, L^{31E}, L^{32A}, L^{32B}, L^{32C}, L^{32D}, L^{32E}, L^{33A}, L^{33B}, L^{33C}, L^{33D}, and L^{33E} is independently a bond, —C(O)NH—, —NHC(O)—, —N(H)—, —O—, —S—, —S—S—, —C(O)—, —NHC(O)NH—, —C(O)O—, —OC(O)—, —(O(CH₂)_{q})ᵣ—, —(NH(CH₂)ₛ)ₜ—, —S—S—(CH₂)_{z}—, substituted or unsubstituted alkylene, or substituted or unsubstituted heteroalkylene;
q is an integer from 1 to 5;
r is an integer from 1 to 250;
s is an integer from 1 to 5;
t is an integer from 1 to 10;
z is an integer from 1 to 10;
each R^{1A}, R^{1B}, R^{1C}, R^{1D}, R^{2A}, R^{2B}, R^{2C}, R^{2D}, R^{3A}, R^{3B}, R^{3C}, R^{3D}, R^{10A}, R^{10B}, R^{10C}, and R^{10D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} bonded to the same nitrogen atom may optionally be joined to form a substituted or unsubstituted heterocycloalkyl or substituted or unsubstituted heteroaryl; R^{2A} and R^{2B} bonded to the same nitrogen atom may optionally be joined to form a substituted or unsubstituted heterocycloalkyl or substituted or unsubstituted heteroaryl; R^{3A} and R^{3B} bonded to the same nitrogen atom may optionally be joined to form a substituted or unsubstituted heterocycloalkyl or substituted or unsubstituted heteroaryl; R^{10A} and R^{10B} bonded to the same nitrogen atom may optionally be joined to form a substituted or unsubstituted heterocycloalkyl or substituted or unsubstituted heteroaryl;
each X, X¹, X², X³, and X¹⁰ is independently —F, —Cl, —Br, or —I;
each n1, n2, n3, and n10 are independently an integer from 0 to 4; and
each v1, v2, v3, and v10 is independently 1 or 2,
provided that at least one of L^{10A}, L^{10B}, L^{10C}, L^{10D}, L^{10E}, L^{11A}, L^{11B}, L^{11C}, L^{11D}, L^{11E}, L^{12A}, L^{12B}, L^{12C}, L^{12D}, L^{12E}, L^{13A}, L^{13B}, L^{13C}, L^{13D}, L^{13E}, L^{20A}, L^{20B}, L^{20C}, L^{20D}, L^{20E}, L^{21A}, L^{21B} L^{21C}, L^{21D}, L^{21E}, L^{22A}, L^{22B}, L^{22C}, L^{22D}, L^{22E}, L^{23A}, L^{23B}, L^{23C}, L^{23D}, L^{23E}, L^{31A}, L^{31B}, L^{31C} L^{31D}, L^{31E}, L^{32A}, L^{32B}, L^{32C}, L^{32D}, L^{32E}, L^{33A}, L^{33B}, L^{33C}, L^{33D}, and L^{33E} is —S—, or —S—S—.

35-48. (canceled)
